(19) United States
(12) Patent Application Publication
(10) Pub. No.: US 2026/0331215 A1
(43) Pub. Date: Oct. 22, 2026

(54) MACHINE LEARNING MODELS TO PREDICT PERFORMANCE OF LIGASES

(71) Applicant: Codexis, Inc., Redwood City, CA (US)

(72) Inventors: Zhe Rui, Redwood City, CA (US); Alexander Bimm, Redwood City, CA (US); Mathew G. Miller, Redwood City, CA (US)

(21) Appl. No.: 19/553,620

(22) Filed: Mar. 2, 2026

Related U.S. Application Data
(60) Provisional application No. 63/767,485, filed on Mar. 5, 2025.

(51) Int. Cl. G16B 15/30; G16B 40/00

(52) U.S. Cl. CPC G16B 15/30; G16B 40/00

(57) ABSTRACT

Disclosed herein are computer-implemented methods that involve structural information charactering a double stranded nucleic acid having one or more chemical modifications of the nucleotides. The methods identify ligation sites on one or both strands of the double stranded nucleic acid. For a given ligation site, a method may identify information about the types of nucleotides located at first defined positions with respect to the current ligation site and/or information about chemical modifications of nucleotides located at second defined positions with respect to the current ligation site. For the ligation site, a method may use a trained computational model to predict, based on the features of the current ligation site, whether the current ligation site meets a criterion for a ligation reaction at the current ligation site with a ligase. The methods may identify a combination of ligation sites to produce the double stranded nucleic acid using the ligase.

### BACKGROUND

Nucleic acid synthesis is conducted using any of various techniques including stepwise nucleoside coupling reactions. Ligation reaction reactions are sometimes used to couple two oligonucleotide fragments to form a product nucleic acid.

Background and contextual descriptions contained herein are provided solely for the purpose of generally presenting the context of the disclosure. Much of this disclosure presents work of the inventors, and simply because such work is described in the background section or presented as context elsewhere herein does not mean that such work is admitted prior art.

### DETAILED DESCRIPTION

The present disclosure provides methods of determining pathways for synthesizing oligonucleotides using ligases. In some embodiments, the methods employ machine learning models that predict one or more characteristics or criteria for ligation reactions.

### Introduction and Context

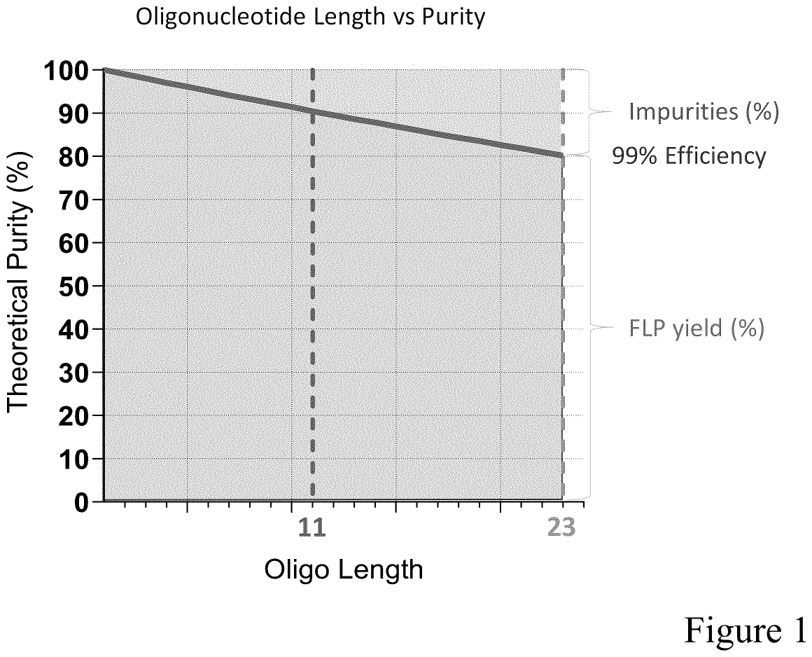

A challenge for stepwise oligonucleotide manufacturing, whether by chemical techniques such as phosphoramidite chemistry or by an enzymatic method, is that coupling at each step is incomplete. Thus, oligonucleotide purity declines with length. See FIG. 1.

With each consecutive cycle, the yield of the full-length product (FLP) decreases while impurities accumulate. A well-optimized phosphoramidite process with 99% coupling efficiency achieves only 80% purity for a 23-mer synthesis, whereas an 11-mer oligonucleotide can achieve upwards of 90% purity due to the smaller number of stepwise additions. Because of these challenges, shorter oligonucleotides have inherent advantages in synthesis, including higher yield, fewer impurities, compatibility with both solid- and liquid-phase phosphoramidite chemistry, simpler purification when necessary, and a reduced risk of batch failure.

One strategy for improving the yield of synthesized nucleic acid products involves chemically synthesizing short oligonucleotide fragments of sizes that can be made in high yield and then ligating these fragments to produce final product in comparably high yield. Such strategy balances the advantages of using short oligonucleotides against the disadvantage of decreasing purity over many coupling steps is a hybrid synthesis strategy. The hybrid strategy may be characterized as (a) chemical synthesis of short oligonucleotide fragments, and (b) ligation of the fragments produced in (a).

In some embodiments, a hybrid synthesis strategy is used to produce double stranded nucleic acids having a length of about 10 to 50 nucleotides, or about 20 to 25 nucleotides, e.g. siRNA molecules. In certain embodiments, the fragments in a hybrid synthesis strategy are about 5 to 30 nucleotides in length, or about 5 to 20 nucleotides in length.

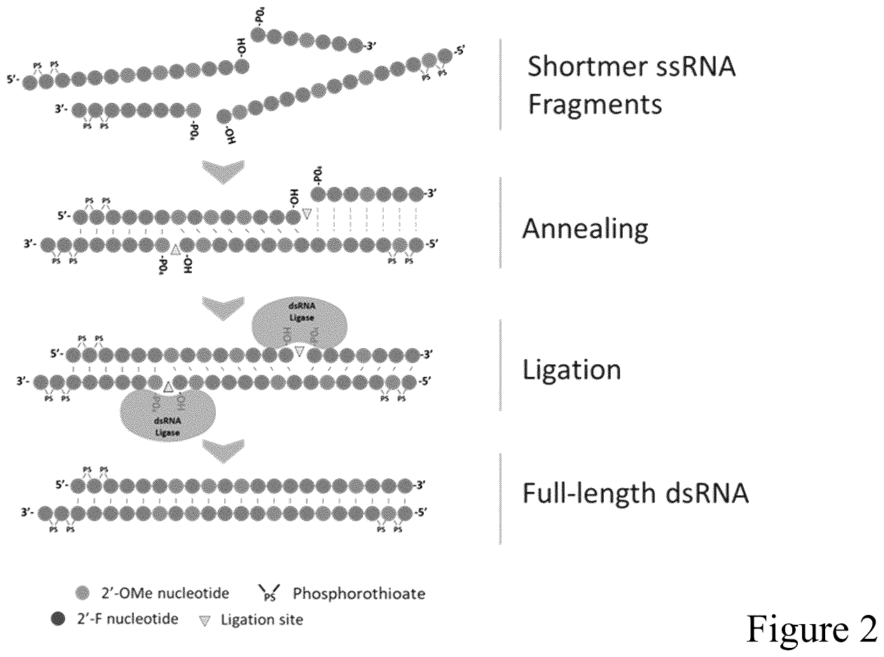

A fragment ligation approach to double stranded RNA synthesis is diagrammed in FIG. 2. Short oligo fragments are synthesized using either chemical or enzymatic methods, with the benefit of their inherent advantages in yield and purity. These oligos are then annealed in solution according to their base complementarity, and a double-stranded RNA ligase is used to join the nicks in the annealed substrate via the 5′ phosphates added during synthesis. The result is a full-length double stranded nucleic that is chemically identical to one generated through conventional synthesis but with a higher overall yield and purity.

Certain aspects of this disclosure pertain to nucleic acid synthesis strategies that (a) identify oligonucleotide fragments of size no greater than can be chemically synthesized to a pre-defined yield and an associated ligation enzyme (ligase) for ligating those fragments; (b) chemically synthesize the segments, and (c) ligate the fragments using an enzyme identified in (a). In designing such strategy, there remains a challenge of identifying suitable RNA fragments and associated ligase.

The success of such a hybrid fragment ligation approach to nucleic acid synthesis depends on the activity of the double-stranded ligase(s) used to combine the fragments through enzymatic ligation. Engineered ligases have been developed with improvements in tolerance to high substrate loads and lower requirements for enzyme loading. However, wild type and engineered ligases both have strong preferences for sequences and modification adjacent to the ligation junction. These preferences limit the substrate range for a given ligase.

Some aspects of this disclosure pertain to computational methods for developing hybrid synthesis strategies by identifying (1) fragments of the target nucleic acid and (2) corresponding ligases to ligate the identified fragments to produce the target nucleic acid.

This disclosure provides synthesis design strategies that employ machine learning models to predict efficient single-nick or multiple (2+) nick fragment designs for which a single ligase can seal all the nicks in the manufacturing of double stranded nucleic acids such as a modified siRNA duplexes. This predictive capability increases the likelihood of successfully designing a double stranded nucleic acid ligation screening project and reduces or eliminates the need to screen a large library of engineered ligases to achieve the process goals.

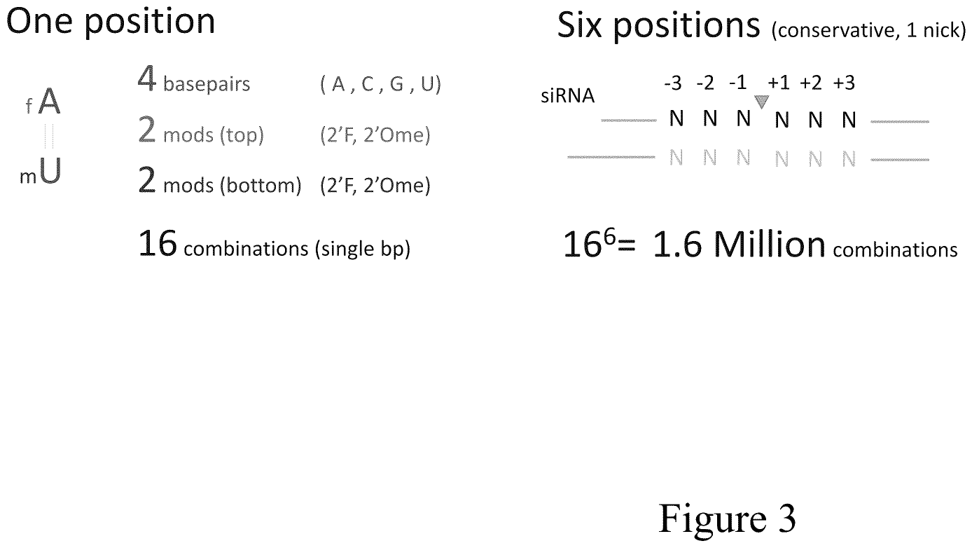

Approved siRNA drug molecules are comprised of almost completely 2′ modified RNA oligonucleotides that enhance their activity and reduce immunogenicity, including 2′-fluoro and 2′-methoxy substitutions. Because there are four nucleobases, and modifications on the top and bottom strand at each position, there are 16 possible combinations at each position in an siRNA duplex. If characteristics of nucleotides at least three positions upstream and downstream of the ligation site can affect the activity of the dsRNA ligase on a given junction, there are 1.6 million possible combinations of sequences and modifications that may affect the activity of the dsRNA ligase. See FIG. 3.

There are many examples of dsRNA ligases (wild type and engineered) with activities that vary based on the sequences and modifications adjacent to the ligation junction. For perspective, the inventors have found that somewhere between 5% to 8% of randomly selected double-nick fragment designs can be converted to greater than 90% efficiency for a given ligase in a one-pot reaction. Improving the odds of a successfully designing a hybrid nucleic acid synthesis strategy may require successfully identifying RNA fragments tailored to a specific dsRNA ligase.

An accurate predictive model reduces the search space to the most likely successful ligases and RNA fragments. This increases the overall success rate in designing RNA synthesis strategies and reduces the time required to screen large numbers of ligases. While there may be some false positives among a predicted set of fragment designs, the hit rate among the predicted fragment designs is much higher than random selection.

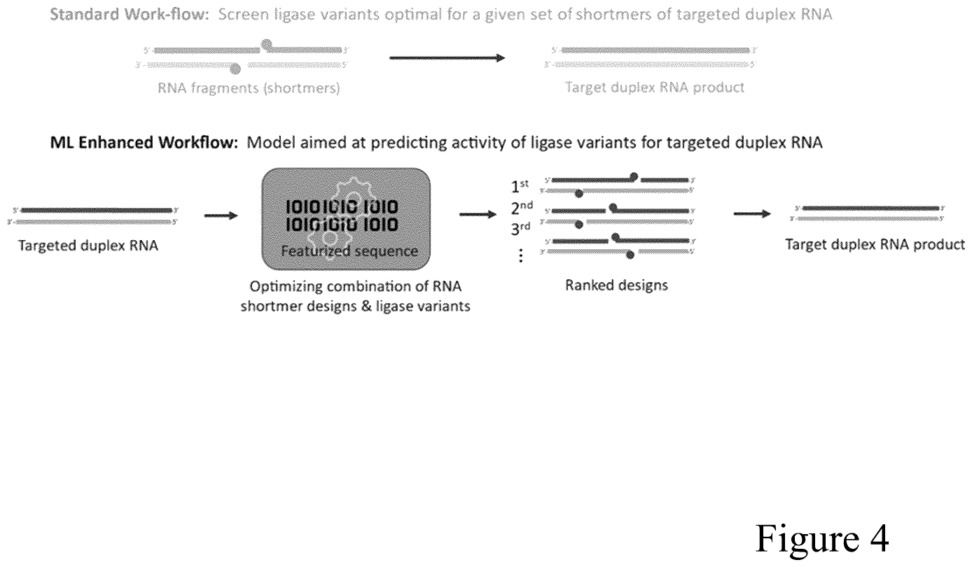

FIG. 4 compares approaches to designing synthesis strategies that employ machine learning models to identify oligomer and ligase pairings and those that do not. Employing accurate machine learning models significantly accelerates lead identification of ligase and short oligomer pairings by maximizing enzyme-nucleic acid compatibility while minimizing screening of large collections on ligase variants and multiple RNA oligomer designs

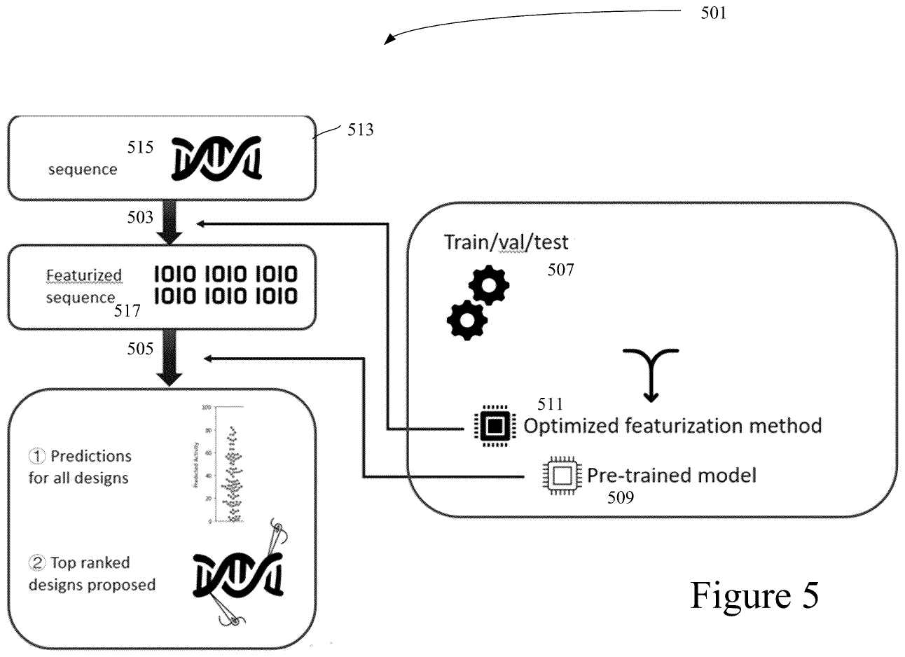

FIG. 5 illustrates an example workflow 501 employing a feature extraction 503 and activity prediction 505 using a machine learning model 509. Before feature extraction and activity prediction can be deployed to guide development of synthesis strategies, they must be generated. The associated training phase is represented in the right-side of workflow 501. Training, validation, and testing processes 507 generate a feature extraction routine 511 and the trained machine learning model 509.

Initially, during the inference phase, during which a synthesis strategy is developed, sequence information 515 of the target nucleic acid is input. See block 513. The sequence information may include information about chemical modifications to nucleotides, secondary structures, etc. The featurization method 511 is then applied to the sequence information 515 to generate a featurized sequence 517. See operation 503.

In the final phase, featurized sequence 517 is input to trained machine learning model 509, which predicts the performance of various synthesis schemes, each of which proposes a different set of ligation points and an associated ligase. Optionally, the workflow also proposes one or a ranked list of synthesis schemes based on predicted performance.

The methods and systems of this disclosure generally pertain to synthesizing any type of target nucleic acid by synthesizing and ligating short nucleic acid fragments. In various embodiments, the target nucleic acid is a double stranded nucleic acid. Included within the concept of a double stranded nucleic acid are double stranded molecules that have one or more single stranded sections or terminal regions. Also, included within the concept of a double stranded nucleic acid are double stranded nucleic molecules that have one or more secondary structures such as hairpins and pseudoknots. In certain embodiments, the double stranded nucleic acid is an RNA molecule. In some cases, the RNA molecule is an siRNA molecule, which may be used for therapeutic applications.

In some embodiments, the nucleic acid has non-natural or variant backbone structure: i.e., a variant of the naturally occurring phosphodiester structure. Some such variations occur in the sugar moiety. Examples include GNAs (glycol nucleic acids) and LNAs (locked nucleic acids). Other variations occur in the phosphate moiety. One example of such variation is a nucleic acid employing a phosphorothioate moiety.

As indicated, an oligonucleotide to be synthesized according to a synthesis scheme as designed herein may be an oligonucleotide therapeutic such as a synthetic single-stranded or double stranded polynucleotide. Such oligonucleotides represent a class of drugs that exert their biological effects by modulating gene expression. The oligonucleotides are designed primarily to target pre-mRNA, mRNA, or non-coding RNA (e.g., miRNA) to promote RNA degradation, modulate splicing, interrupt translation, or in some instances activate expression. Small interfering RNAs (siRNA) are a class of oligonucleotide therapeutics that are typically double stranded oligonucleotides that act via RISC (RNA-induced silencing complex) pathway, where the strand complementary to an RNA target, also referred to as the guide strand, targets the RNA for degradation or translation inhibition. Antisense oligonucleotides (ASOs) are single-stranded and designed to bind sequence specifically to a target RNA and modulate protein expression via several different mechanisms. Targets of ASOs include pre-mRNA, mRNA, and non-coding RNA.

siRNA and ASO oligonucleotides are designed to include various modifications to improve in vivo stability, cellular delivery, specificity, and efficacy. Sugar modifications include 2′-O-methyl, 2′-O-ethyl, 2′-O-methoxyethyl, 2′-fluoro, and locked nucleic acid (LNA); modification of internucleoside linkages include phosphorothioate and phosphoramidate morpholino linkages; and nucleobase modifications include 5′-methylcytosine and G-clamp. In addition, conjugating targeting moieties to the oligonucleotide, such as GalNac and lipid groups, can enhance delivery to cells and tissues.

### Abbreviations and Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, the singular forms “a”, “an” and “the” include plural referents unless the context clearly indicates otherwise. Thus, for example, reference to “a polypeptide” includes more than one polypeptide.

Similarly, “comprise,” “comprises,” “comprising” “include,” “includes,” and “including” are interchangeable and not intended to be limiting. Thus, as used herein, the term “comprising” and its cognates are used in their inclusive sense (i.e., equivalent to the term “including” and its corresponding cognates).

It is to be further understood that where descriptions of various embodiments use the term “comprising,” those skilled in the art would understand that in some specific instances, an embodiment can be alternatively described using language “consisting essentially of” or “consisting of.”

“About” means an acceptable error for a particular value. In some instances, “about” means within 0.05%, 0.5%, 1.0%, or 2.0%, of a given value range. In some instances, “about” means within 1, 2, 3, or 4 standard deviations of a given value.

“Ligation site” refers to a location in a nucleic acid sequence where a ligation reaction will be performed to remove a nick by forming a covalent bond that between adjacent nucleotides. A ligation site may represent the terminal positions of two nucleic acid fragments that are to be joined by the ligation reaction. In naturally occurring and some other types of nucleic acids, a ligation reaction forms a phosphodiester bond at the ligation position. Ligation site is also sometimes referred to as “nick position,” “ligation position,” or “ligation end.”

“Feature” refers to something that serves to describe or identify a physical component or property. For example, characters representing nucleotides in a nucleic acid are features. In another example, identifications of chemical modifications to nucleotides are features. Feature can also refer to properties of items, such as the activity of an enzyme on a particular substrate, or the selectivity or solvent stability of a nucleic acid.

A “feature extractor” is a computational module configured to receive input information (e.g., sequence and chemical modification information about a nucleic acid) and output a transformed representation of the information, which may be suitable as input to a trained machine learning model. In some embodiments, the transformed representation has reduced dimensionality in comparison to the input information.

A “machine learning model” may be any trained computational model. In some embodiments herein, a machine learning model is configured to receive as inputs features characterizing a target nucleic acid, potential ligation positions within the nucleic acid, ligases, chemical structural details of nucleotides within the nucleic acid, etc. Examples of machine learning models include neural networks, including recurrent neural networks, convolutional neural networks and generative adversarial networks, autoencoders, including variational autoencoders, random forests models, restricted Boltzmann machines, gradient boosted trees, gaussian process regression, PLS (Partial Least Squares) models, support vector machines, support vector regressions, models produced by regularized techniques such as Ridge and Lasso regressions, attention-based models such as a transformer models, and probabilistic graphical models, including Bayesian networks. In some embodiments herein, machine learning models are trained using a training set that reflects a range of sequence information for oligonucleotides employed in ligation reactions for which the model should be able to accurately predict characteristics of the ligation reaction such as efficiency or yield.

In general, though not necessarily, a neural network includes multiple layers. Each such layer may include multiple processing nodes, and the layers process in sequence, with nodes of layers closer to the model input layer processing before nodes of layers closer to the model output. In various embodiments, one layers feeds to the next, etc. The output layer may include one or more nodes configured to output information representing chemical composition and/or perceptual characteristics of a finished wine.

In some embodiments, the model has more than two (or more than three or more than four or more than five or more than six) layers of processing nodes that receive values from preceding layers (or as direct inputs) and that output values to succeeding layers (or the final output). Interior nodes are often “hidden” in the sense that their input and output values are not visible outside the model. In various embodiments, the operation of the hidden nodes need not be monitored or recorded during operation.

In some implementations, a machine learning model is trained using an unsupervised learning method. In some implementations, a machine learning model is trained using a supervised learning method. In some implementations, a machine learning model is trained using a semi-supervised learning method. For example, the training is performed using a set of training data, but only subset of that data has labels. In various implementations, generative adversarial networks, variational autoencoders, or other types of autoencoders are trained using semi-supervised learning methods.

“Training data” refers to a set of data that is employed in a machine learning training algorithm to produce a machine learning model. In supervised and semi-supervised training algorithms, training data includes features and labels. As an example, ligation sites together with nucleotide sequence and modification data proximate the ligation site may comprise features, while ligation reaction characteristics at the ligation site (e.g., coupling efficiency) may comprise labels.

“Predictive power” refers to the ability of a model to correctly predict the values of a dependent variable for data under various conditions. For example, the predictive power of a model refers to the ability of the model to predict characteristics of a ligation reaction from, inter alia, sequence information about fragments to be ligated by the ligation reaction.

The terms “regression” and “regression analysis” refer to techniques used to understand which of the independent variables are related to the dependent variable, and to explore the forms of these relationships. In restricted circumstances, regression analysis can be used to infer causal relationships between the independent and dependent variables. It is a statistical technique for estimating the relationships among variables. Regression techniques may model and/or analyze several variables, while focusing on the relationship between a dependent variable and one or more independent variables.

“Partial Least Squares” (“PLS”) refers to a family of methods that finds a linear regression model by projecting predicted variables (e.g., ligation performance) and the observable variables (e.g., sequences) to a new space. PLS is also known as “projection to latent structures.” Both the X (independent variables) and Y (dependent variables) data are projected to new spaces. PLS is used to find the fundamental relations between two matrices (X and Y). A latent variable model is used to model the covariance structures in the X and Y spaces. A PLS model will try to find the multi-dimensional direction in the X space that explains the maximum multi-dimensional variance direction in the Y space. PLS regression is particularly useful when the matrix of predictors has more variables than observations, and when there is multi-collinearity among X values.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably to denote a polymer of at least two amino acids covalently linked by an amide bond, regardless of length or post-translational modification (e.g., glycosylation or phosphorylation).

“Amino acids” and “amino acid” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glycine (Gly or G), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about a-carbon (Cₐ). For example, whereas “Ala” designates alanine without specifying the configuration about the a-carbon, “D-Ala” and “L-Ala” designate D-alanine and L-alanine, respectively. When the one-letter abbreviations are used, upper case letters designate amino acids in the L-configuration about the a-carbon and lower-case letters designate amino acids in the D-configuration about the a-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein,” and “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“RNA ligase” refers to enzymes that covalently joins the 5′-phosphoryl termini of RNA or DNA to the 3′-hydroxyl termini of RNA or DNA. Families of known RNA ligases include RNA ligase 1, also referred to as single-stranded RNA ligase or ssRNA ligase, which catalyzes the covalent joining of single-stranded 5′-phosphoryl termini of RNA or DNA to single-stranded 3-hydroxyl termini of RNA or DNA. RNA ligase 2, also referred to as double stranded RNA ligase or dsRNA ligase, also catalyzes the covalent joining of a 3′-hydroxyl terminus of RNA to a 5′-phosphorylated RNA or DNA but shows preference for double stranded substrates. In some embodiments, RNA ligases include those enzymes classified in EC 6.5.1.3. It is to be understood that the ligation reaction is not limited to naturally occurring RNA and DNA substrates also includes nucleotide substrates that contain modified nucleotides and/or nucleotide analogs.

“Polynucleotide,” “nucleic acid,” or “oligonucleotide” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When a polynucleotide, nucleic acid, or oligonucleotide sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid. The polynucleotide or nucleic acid may be single-stranded or double-stranded, or may include both single-stranded regions and double-stranded regions.

In some embodiments, the terms “polynucleotide,” “nucleic acid” and “oligonucleotide” encompass polynucleotide or nucleic acid or oligonucleotide analogs or modified polynucleotide or nucleic acid or oligonucleotide, which include, among others, nucleosides linked together via other than standard phosphodiester linkages, such as non-standard linkages of phosphoramidates, phosphorothioates, amide linkages, etc.; nucleosides with modified and/or synthetic nucleobases, for example inosine, xanthine, hypoxanthine, etc.; nucleosides with modified sugar residues, such as 2′-O-alkyl, 2′-halo, 2,3-dideoxy, 2′-halo-2′-deoxy, □-D-ribo LNA, □-L-ribo-LNA (e.g., locked nucleic acids), etc.; and/or 5′-phosphate analogs, including, among others, phosphorothioate, phosphoacetate, phosphoramidate, monomethylphosphate, methylphosphonate, or phosphonocarboxylate.

“Nucleobase” refers to means an unmodified nucleobase or a modified nucleobase. As used herein an “unmodified nucleobase” is adenine (A), thymine (T). cytosine (C). uracil (U). or guanine (G). A “modified nucleobase” refers to a group of atoms other than unmodified A, T, C, U. or G capable of pairing with at least one unmodified nucleobase.

“Nucleoside” refers to a compound comprising a nucleobase and a sugar moiety. The nucleobases and sugar moiety are each, independently, unmodified or modified.

“Internucleoside linkage” refers to as a linkage that covalently couples two nucleosides together. In the oligonucleotides herein, internucleoside linkages covalently couple adjacent nucleosides together, typically forming a bond between the sugar moieties of the adjacent nucleosides. Non-limiting examples of internucleoside linkages include phosphodiester —O—P(O)₂—O— linkages and modified internucleoside linkages, such as phosphorothioate —O—P—(O, S)—O— and phosphorodithioate —O—P(S)₂—O—.

“Modified oligonucleotide” refers to an oligonucleotide which contains at least one modified internucleoside linkage and/or a modified nucleoside, or a modified terminal group.

“Modified nucleotide” refers to a nucleotide (e.g., NMP, NDP, NTP) in which at least one of the phosphate is a modified phosphate group and/or a modified nucleoside.

“Modified nucleoside” or “nucleoside modification” refers to a nucleoside modified as compared to the equivalent DNA or RNA nucleoside by the introduction of one or more modifications of the sugar moiety or the nucleobase. The modified nucleoside comprises a modified nucleobase and/or a modified sugar residue. The term “modified nucleoside” may also be used herein interchangeably with the term “nucleoside analogue.” Nucleosides with an unmodified DNA or RNA sugar moiety are termed DNA or RNA nucleosides herein. Nucleosides with modifications in the nucleobase of the DNA or RNA nucleoside are still generally termed DNA or RNA if they allow Watson-Crick base pairing.

“Modified internucleoside linkage” refers to as a linkage other than a phosphodiester (PO) linkage that covalently connects two nucleosides together. In some embodiments, exemplary modified internucleoside linkage is a phosphorothioate or phosphorodithioate internucleoside linkage. Other modified phosphorus-containing internucleoside linkages include phosphotriesters, methylphosphonates, and phosphoramidates (P—NH₂). See, e.g., Clave et al., RSC Chem Biol., 2021 2(1): 94-150). In some embodiments, the modified internucleoside linkage is a non-phosphorus containing internucleoside linkage, including but not limited tomethylenemethylimino (—CH₂—N(CH₃)—O—CH₂), thiodiestcr, thionocarbamate (—O—C(═O)(NH)—S—); siloxane (—O—SiH₂—O—); N,N′-dimethylhydrazine (—CH₂—N((CH₃)—N((CH₃)—); MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). In some embodiments, internucleoside linkages having a chiral atom can be prepared as a mixture of the stereoisomers, or as separate stereoisomers.

“Phosphorothioate internucleoside linkage” refers to an internucleoside linkage in which one of the oxygen atom in a phosphodiester linkage is replaced with a sulfur atom. In some embodiments, a phosphorothioate linkage may be represented as —O—P(O,S)—O—, wherein one of the non-bridging oxygen atoms is replaced with a sulfur atom. Phosphorothioate internucleoside linkages are chiral (see, for example, Jahns et al. 2022, Nucleic Acids Research Vol. 50, No 3, 1221-1240), with right-handed (Rp) and left-handed (Sp) isomers. In some embodiments, the Rp diastereomer may be referred to as an R—PS internucleoside linkage or an srP internucleoside linkage. The Sp diastereomer may be referred to as an S—PS internucleoside linkage or ssP internucleoside linkage. In some embodiments, the oligonucleotide comprises one or more srP internucleoside linkages. In some embodiments, the oligonucleotide comprises one or more ssP internucleoside linkages. Where the chirality of a phosphorothioate internucleoside linkage is not specified, that phosphorothioate internucleoside linkage may be either an srP linkage or an ssP linkage.

“Non-bridging phosphorothioate internucleoside linkage” refers to a phosphorothioate internucleoside linkage in which the sulfur atom attached to the phosphorous atom is in place of a non-bridging oxygen atom.

“Non-bridging phosphorodithioate internucleoside linkage” refers to a modified internucleoside linkage which is a non-bridging phosphorodithioate internucleoside linkage. A non-bridging phosphorodithioate internucleoside linkage has two identical sulfur atoms attached to the phosphorous atom, achieved by replacing the non-bridging oxygen atom in the phosphorothioate linkage with a sulfur atom.

“Abasic sugar moiety” refers to a sugar moiety of a nucleoside that is not attached to a 12rylthiol12. In some embodiments, such abasic sugar moieties are referred to as “abasic nucleoside.”

“Inverted nucleoside” refers to a nucleotide having a 3′ to 3′ and/or 5′ to 5′ internucleoside linkage. Similarly, and “inverted sugar moiety” refers to the sugar moiety of an inverted nucleoside or an abasic sugar moiety having a 3′ to 3′ and/or 5′ to 5′ internucleoside linkage.

“LNA nucleoside” or “locked nucleoside” refers to 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non-limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 2002, 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

“Terminal group” as used herein refers to a group located at the first or last nucleoside in a polynucleotide or oligonucleotide. A 5′-terminal group refers to the terminal group bonded to 5′- or 4′-carbon atom of the first nucleoside within a polynucleotide. A 3′-terminal group is a terminal group bonded to 3′-carbon atom of the last nucleoside within a polynucleotide or oligonucleotide.

“5′-blocking group” as used herein refers to a moiety or chemical group that prevents or inhibits attachment of another nucleoside, nucleotide or oligonucleotide to the 5′-terminal nucleoside. In context enzymes active on the 5′-terminal nucleoside, a 5′-blocking group prevents or inhibits the enzyme(s) from attachment of another nucleoside, nucleotide or oligonucleotide to the to the 5′-terminal nucleoside, particularly the 5′-OH of the 5′-terminal nucleoside.

“3′-blocking group” refers to moiety or chemical group that prevents or inhibits attachment off another nucleoside, nucleotide, or oligonucleotide to the 3′-terminal nucleoside. In context of single-stranded RNA ligase or other enzymes active on the 3′-terminal nucleoside, a 3′-blocking group prevents or inhibits the enzyme(s) from attachment of another nucleoside, nucleotide, or oligonucleotide to the 3′-terminal nucleoside, particularly the 3′-OH of the 3′-terminal nucleoside.

“Reversible blocking group” refers to a blocking group that can be removed or cleaved off to provide a free 3′-OH. In some embodiments, the blocking group is removable with a deblocking agent, which can be a chemical or enzymatic deblocking agent.

“Enzymatically reversible blocking group” refers to a blocking group that is susceptible to removal or cleaving by an enzyme.

“Duplex” and “ds” refer to a double-stranded nucleic acid (e.g., DNA or RNA) molecule comprised of two single-stranded polynucleotides that are complementary in their sequence (e.g., A pairs to T or U, C pairs to G), arranged in an antiparallel 5′ to 3′ orientation, and held together by hydrogen bonds between the nucleobases (e.g., adenine [A], guanine [G], cytosine [C], thymine [T], uridine [U]).

“Complementary” is used herein to describe the structural relationship between nucleotide bases that are capable of forming base pairs with one another. For example, a purine nucleotide base present on a polynucleotide that is complementary to a pyrimidine nucleotide base on a polynucleotide may base pair by forming hydrogen bonds with one another. Complementary nucleotide bases can base pair via Watson/Crick base pairing or in any other manner than forms stable duplexes or other nucleic acid structures.

“Watson/Crick Base-Pairing” refers to a pattern of specific pairs of nucleobases and analogs that bind together through sequence-specific hydrogen-bonds, e.g., A pairs with T or U, and G pairs with C.

“Annealing” or “Hybridization” refers to the base-pairing interactions of one nucleobase polymer (e.g., poly- and oligonucleotides) with another that results in the formation of a double-stranded structure, a triplex structure or a quaternary structure. Annealing or hybridization can occur via Watson-Crick base-pairing interactions, but may be mediated by other hydrogen-bonding interactions, such as Hoogsteen base pairing. In some embodiments, the nucleobase polymer that anneals or hybridizes to another is a single nucleobase polymer while in other embodiments, the nucleobase polymers are separate nucleobase polymers.

“Engineered,” “recombinant,” “non-naturally occurring,” and “variant,” when used with reference to a cell, a polynucleotide or a polypeptide refer to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” and synonymously “encoding” refers to that part of a nucleic acid (e.g., a gene) that encodes an amino acid sequence of a protein.

“Percent (%) sequence identity” refers to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (see, e.g., Altschul et al., J. Mol. Biol., 1990, 215: 403-410; and Altschul et al., Nucleic Acids Res., 1977, 3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (see Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to”, “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of a recombinant primase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. In some embodiments, the sequence is tagged (e.g., with a histidine tag).

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence.

“Suitable reaction conditions” or “suitable conditions” refers to those conditions in the enzymatic conversion reaction solution (e.g., ranges of enzyme loading, substrate loading, temperature, pH, buffers, co-solvents, co-factors, etc.) under which an RNA ligase is capable of attaching a nucleotide donor to a nucleotide acceptor at a ligation site. Exemplary “suitable reaction conditions” are provided herein (see, the Examples).

“Product” in the context of an enzymatic conversion process refers to the compound or molecule resulting from the action of the primase polypeptide on the substrate.

“Culturing” refers to the growing of a population of cells under suitable conditions using any suitable medium (e.g., liquid, gel, or solid).

“Produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Heterologous” or “recombinant” refers to the relationship between two or more nucleic acid or polypeptide sequences (e.g., a promoter sequence, signal peptide, terminator sequence, etc.) that are derived from different sources and are not associated in nature.

“Host cell” and “host strain” refer to suitable hosts for expression vectors comprising a polynucleotide provided herein (e.g., a polynucleotide sequences encoding a recombinant primase). In some embodiments, the host cells are prokaryotic or eukaryotic cells that have been transformed or transfected with vectors constructed using recombinant DNA techniques, and progeny thereof, as known in the art.

“Optional” or “optionally” refers to a described event or circumstance may or may not occur, and that the description includes instances where the event or circumstance occurs and instances where the event or circumstance does not. For example, “optionally substituted alkyl” refers to an alkyl group that may or may not be substituted and that the description encompasses both substituted alkyl group and unsubstituted alkyl group.

“Substituted” as used herein means one or more hydrogen atoms of the group is replaced with a substituent atom or group commonly used in pharmaceutical chemistry. Each substituent can be the same or different. Examples of suitable substituents include, but are not limited to, alkyl, alkenyl, alkynyl, cycloalkyl, aryl, arylalkyl, heterocycloalkyl, heteroaryl, OR′ (e.g., hydroxyl, alkyloxy (e.g., methoxy, ethoxy, and propoxy), aryloxy, heteroaryloxy, arylalkyloxy, ether, ester, carbamate, etc.), hydroxyalkyl, alkyloxycarbonyl, alkyloxyalkyloxy, perhaloalkyl, alkyloxyalkyl, SR′ (e.g., thiol, alkylthio, 19rylthiol, heteroarylthio, arylalkylthio, etc.), S+R′₂, S(O)R′, SO₂R′, NR′R′ (e.g., primary amine (i.e., NH₂), secondary amine, tertiary amine, amide, carbamate, urea, etc.), hydrazide, halo, nitrile, nitro, sulfide, sulfoxide, sulfone, sulfonamide, thiol, carboxy, aldehyde, keto, carboxylic acid, ester, amide, imine, and imide, including seleno and thio derivatives thereof, wherein each of the substituents can be optionally further substituted. In embodiments in which a functional group with an aromatic carbon ring is substituted, such substitutions will typically number less than about 10 substitutions, more preferably about 1 to 5, with about 1 or 2 substitutions being preferred.

“Stereoisomer” refers to a compound made up of the same atoms bonded by the same bonds but having different three-dimensional structures, which are not interchangeable. Thus, “stereoisomer thereof” with respect to a compound includes any stereoisomer of the compound and mixtures of stereoisomers, and includes “enantiomers,” which refers to two stereoisomers whose molecules are nonsuperimposable mirror images of one another. A compound may have more than one chiral center such that the compound may exist as either an individual diastereomer or as a mixture of diastereomers.

### Features

For purposes of using a machine learning model to predict a characteristic of a ligation reaction, features that may be input to the machine learning model may include many characteristics relevant to the ligation reaction.

Some features describe one or more ligation position(s) on a nucleic acid to be produced by a ligation reaction. Ligation positions are sometimes referred to as nick positions. Ligation positions may define or imply two more nucleic acid fragments that will be ligated by the ligation reaction. A feature describing a ligation position may be represented in many different ways. For example, it may be represented by a number of nucleotides offset from an end point. Or it may be represented by one or more nucleotide sequences in the vicinity. For example, it may be represented by nucleotide sequences over a defined number of nucleotides upstream and/or downstream of the ligation position.

Other features may identify the number of ligation positions on a nucleic acid strand. For example, a strand of a double stranded target nucleic acid may have none, one, two, or more ligation positions. In some cases, all ligation positions on a single strand are ultimately ligated by a single ligase.

Other features may identify an offset or distance between two ligation positions, one on a first strand and the other on an opposite strand of a double stranded nucleic acid.

Some features identify nucleotides in a sequence of a fragment or a region of a fragment that may participate in a ligation reaction. Such features may identify nucleotides various ways. One approach simply identifies the nucleotide bases present in a sequence: e.g., A, U, C, G for RNA. Another approach identifies the type of base (purine versus pyrimidine) and/or the nucleotide pairing: e.g., A-U versus C-G.

Some features describe chemical modifications of nucleotides with respect to the chemical structures in naturally occurring nucleic acids. Such modifications may be made with respect to the sugar moiety, the phosphate, the nitrogenous base, or any combination thereof. A feature representing a chemical modification of a sugar may identify a substitution position on a sugar and/or a substitution moiety. In some cases, a feature representing a chemical modification with respect to a sugar may identify a complete replacement of the ribose in naturally occurring nucleic acids. Examples of non-ribose replacements include those in GNAs (glycol-based nucleic acids) and LNAs (locked nucleic acids).

Still other features may describe variations of or replacements of phosphate moieties present in naturally occurring nucleic acids. Examples include phosphorothioates, phosphorodithioates, phosphonates, etc.

Chemical modifications that may be relevant to predicting characteristics of ligation reactions, and hence may be featurized, are discussed in more detail here.

### Modified Nucleotides

In some embodiments, the modifications occur on the nucleobase, the sugar moiety, terminal groups, or any combination thereof. The various modifications that can be used are described below.

### 2′- and 3′-Sugar Modifications

In some embodiments, a oligonucleotide comprises a modified nucleoside, wherein the modification is on the sugar moiety of the nucleoside. In some embodiments, the modified sugar moiety is a modified furanosyl sugar moiety, for example ribose or deoxyribose. In some embodiments, the furanosyl sugar moiety is modified or substituted at the 2′, 3′, or a combination of 2′ and 3′ positions. Modifications at the 4′, and/or 5′-positions are described below as “terminal group.”

In some embodiments, the modification is at the 2′-position of the sugar moiety. In some embodiments, substitutions at the 2′-position include, among others, halo (e.g., Cl, F, Br, etc.) or —O— alkyl or 2′-alkoxy (e.g., O-methyl, O-ethyl, etc.). In some embodiments, other modifications at the 2′-position include, but are not limited to, allyl, amino, azido, SH, CN, OCN, CF₃, OCF₃, SCH₃, SOCH₃, SO₂CH₃, ONO₂, NO₂, N₃, and NH₂. In some embodiments, substituent groups at the 2′-position include, among others, O—(C₁-C₁₀)alkoxy, alkoxyalkyl, O-alkyl, S-alkyl, N-alkyl, O-alkenyl, S-alkenyl, N-alkenyl, O-alkynyl, S-alkynyl, N-alkynyl, O-alkyl-O-alkyl, alkynyl, wherein the alkyl, alkenyl and alkynyl can be substituted or unsubstituted C₁-C₁₀ alkyl or C₁-C₁₀ alkenyl and alkynyl. In some embodiments, substituent groups at the 2′-position include, but are not limited to, alkaryl, aralkyl, O-alkaryl, and O-aralkyl. In some embodiments, the substitution at the 2′-position is a phosphate (see, e.g., Current Protocols in Nucleic Acid Chemistry, 13.1.1-13.1.31, John Wiley & Sons (2003).

In some embodiments, the modified 2′-position of the sugar moiety is halo, 2′-O—R′, or 2′-O—COR′, where R′ is an alkyl, alkyloxyalkyl, cycloalkyl, heterocyclyl, aryl, heteroaryl, cycloalkylalkyl, heterocyclylalkyl, arylalkyl, or heteroarylalkyl. In some embodiments, R′ is a C₁-C₄alkyl. In some embodiments, the modified 2′-position is a 2′-O—R′, wherein in R′ is alkyloxyalkyl, alkylamine, cyanoalkyl, or —C(O)-alkyl. In some embodiments, the 2′-position of the sugar moiety of the nucleoside substrate is —O—R′, wherein R′ is —CH₃ or —CH₂CH₃ or —CH₂CH₂OCH₃. In some embodiments, the modified 2′-position is 2′-O-(2-methoxyethyl), 2′-O-allyl, 2′-O-propargyl, 2′-O-ethylamine, 2′-O-cyanoethyl, -2′-O-amine, or 2′-O-acetate ester.

In some embodiments, a modification at the 2′-position comprises a locked nucleoside. In some embodiments, locked nucleosides comprises a biradical linking the C2′ and C4′ of the ribose sugar ring of said nucleoside (also referred to as a “2′-4′ bridge”), which restricts or locks the conformation of the ribose ring (see, e.g., Obika et al., Tetrahedron Letters, 1997, 38(50):8735-8738; Orum et al., Current Pharmaceutical Design, 2008, 14(11):1138-1142). In some embodiments, the ribose moiety of the locked nucleotide is in the C3′-endo (beta-D) or C2′-endo (alpha-L) conformation. In some embodiments, the bridge is a methylene bridge. In some embodiments, the bridge is an ethylene bridge, also referred to as ENA (see, e.g., Morita et al., Bioorg Med Chem Lett., 2002, 12(1):73-6). Other locked nucleoside are described in International patent publication WO 2121249993, incorporated by reference herein.

In some embodiments, other locked nucleosides include, among others, 5′-methyl-LNA, 2′-amino-LNA, alpha-L-LNA, and thio-LNA. Structures of certain locked nucleosides are shown below:

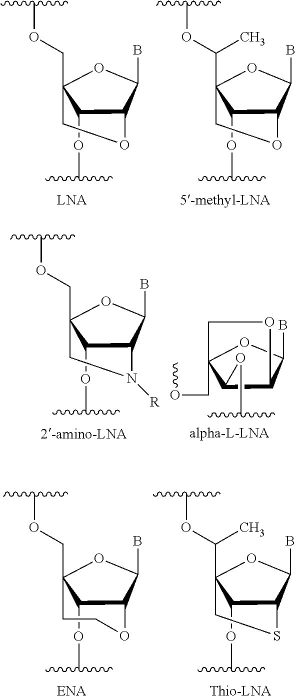

where R in the above is alkyl or acyl.

In some embodiments, a modification at the 2′-position comprises a reactive moiety; a conjugate moiety, including a conjugate moiety attached via a linker or a linker, as described herein.

In some embodiments, the modification is at the 3′-position of the sugar moiety. In some embodiments, in view of the effect of a 3′-modification on ligase activity, and use of the 3′-OH group for internucleoside linkage, the 3′-modification is on the 3′-terminal nucleoside of the nucleotide donor. In some embodiments, the modification at the 3′-position are similar to those at the 2′-position. In some embodiments, substitutions at the 3′-position include, among others, halo (e.g., Cl, F, Br, etc.) or —O— alkyl or 3′-alkoxy (e.g., O-methyl, O-ethyl, etc.). In some embodiments, other modifications at the 3′-position include, but are not limited to, allyl, amino, azido, SH, CN, OCN, CF₃, OCF₃, SCH₃, SOCH₃, SO₂CH₃, ONO₂, NO₂, N₃, and NH₂. In some embodiments, substituent groups at the 3′-position include, among others, O—(C₁-C₁₀)alkoxy, alkoxyalkyl, O-alkyl, S-alkyl, N-alkyl, O-alkenyl, S-alkenyl, N-alkenyl, O-alkynyl, S-alkynyl, N-alkynyl, O-alkyl-O-alkyl, alkynyl, wherein the alkyl, alkenyl and alkynyl can be substituted or unsubstituted C₁-C₁₀ alkyl or C₁-C₁₀ alkenyl and alkynyl. In some embodiments, substituent groups at the 3′-position include, but are not limited to, alkaryl, aralkyl, O-alkaryl, and O-aralkyl. In some embodiments, the substitution at the 3′-position is a phosphate.

In some embodiments, the modified 3′-position of the sugar moiety is halo, 3′-O—R′, or 3′-O—COR′, where R′ is an alkyl, alkyloxyalkyl, cycloalkyl, heterocyclyl, aryl, heteroaryl, cycloalkylalkyl, heterocyclylalkyl, arylalkyl, or heteroarylalkyl. In some embodiments, R′ is a C₁-C₄alkyl. In some embodiments, the modified 3′-position is a 3′-O—R′, wherein in R′ is alkyloxyalkyl, alkylamine, cyanoalkyl, or —C(O)-alkyl. In some embodiments, the 3′-position of the sugar moiety of the nucleoside substrate is —O—R′, wherein R′ is —CH₃ or —CH₂CH₃ or —CH₂CH₂OCH₃. In some embodiments, the modified 3′-position is 3′-O-(2-methoxyethyl), 3′-O-allyl, 3′-O-propargyl, 3′-O-ethylamine, 3′-O-cyanoethyl, -3′-O-amine, or 3′-O-acetate ester.

In some embodiments, the modifications at the 3′-position is a reversible or cleavable 3′-blocking group. In some embodiments, removal or cleaving of the reversible or cleavable 3′-blocking group results in a free 3′-OH group, which in some embodiments can serve as an acceptor for single-stranded RNA ligase or a terminal nucleotidyl transferase. In some embodiments, exemplary reversible or cleavable 3′-blocking groups include, among others, 3′-O-azidomethyl, 3′-O-(2-methoxyethyl), 3′-O-allyl, 3′-O-propargyl, 3′-O-ethylamine, 3′-O-cyanoethyl, -3′-O-amine, 3′-O-acetate ester, 3′-phosphate, 3′-diphosphate, or 3′-triphosphate. In some embodiments, the 3′-blocking group is paired with the corresponding deblocking agent used in the deblocking or cleavage of the 3′-blocking group. Other reversible or cleavable 3′-blocking groups are described in International patent publication WO2023183569, incorporated by reference herein.

In some embodiments, a modification at the 3′-position comprises a reactive moiety; a conjugate moiety, including a conjugate moiety attached via a linker′ or a linker, as described herein.

In some embodiments, the modified sugar moiety comprises an unlocked nucleoside. In some embodiments, in the unlocked nucleoside, the furanosyl ring is opened to result in the structure below:

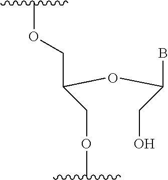

where B represents the nucleobase. Unlocked nucleosides are described in, among others, International patent publication WO2022/098990 and Snead et al., Molecular Therapy-Nucleic Acids, 2013, 2, e103.

### Modified Nucleobases

In some embodiments, the oligonucleotide comprises one or more nucleosides comprising a modified nucleobase. In some embodiments, modified nucleobase that is capable of hydrogen bonding to form Watson and Crick type base pairing is selected. In some embodiments, some positions for possible modifications on naturally occurring nucleobases are indicated below:

| Nucleobase | Modification Positions |
|---|---|
| 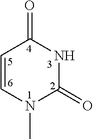 | N1, C2 ═O, N3, C4 ═O, C5 |
| Uridine | |
| 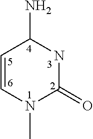 | C2 ═O, N3, C4 NH₂, C5, |
| Cytidine | |
| 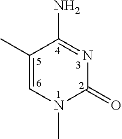 | C2 ═O, N3, C4 NH₂ |
| Thymidine | |
| 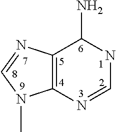 | N1, C2, C6 NH₂, C8 |
| Adenosine | |
| 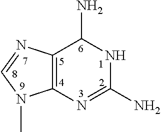 | N1, C2 NH₂, N7 |
| Guanosine | |

In some embodiments, the nucleobase comprise an inosine nucleoside (i.e., nucleosides comprising a hypoxantine nucleobase). Ln some embodiments, the modified nucleobase is 5-substituted pyrimidines, 6-azapyrimidines, alkyl or alkynyl substituted pyrimidines, alkyl substituted purines, and N-2. N-6 and O-6 substituted purines. In some embodiments, the modified nucleobase is 2-aminopropyladenine. 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine, and 2-thiocytosine. 5-propynyl uracil, 5-propynylcytosine. 6-azouracil, 6-azocytosine, 6-azothymine. 5-ribosyluracil (pseudouracil), 4-thiouracil. 8-halo purine, 8-amino purine, 8-thio purine, 8-thioalkyl purine, 8-hydroxy purine, 8-aza purine, 5-bromocytosine. 5-trifluoromethylcytosine, 5-halouracil, 5-halocytosine, 7-methylguanine, 7-methyladenine, 2-F-adenine, 2-aminoadenine, 7-deazaguanine, 7-deazaadenine. 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, and 5-methyl 4-N-benzoyluracil. Further modified nucleobases include tricyclic pyrimidines, e.g., 1,3-diazaphenoxazine-2-one. 1,3-diazaphenothiazine-2-one, and 9-(2-aminoethoxy)-1,3-diazaphenoxazine-2-one (G-clamp).

In some embodiments, the modified nucleobase includes, among others, nucleobases based on 2,4-dihalotolene and benzimidazole groups. In some embodiments, the modified nucleobase is 4-methylbenzimidazole, 2,4-difluorotoluene, 9-methylimidazo[(4,5)-b]pyridine, 2,4-dibromotoluene, benzimidazole, 5-nitrobenzimidazole, 6-nitrobenzimidazole, and 5-nitroindole. In some embodiments, the modified nucleobase is 7-azaindole, and isocarbostyril (see, e.g., Berdis et al., Front. Chem. 10:1051525). Other modified nucleobases are described in, among others, patent publication WO2021249993.

In some embodiments, included within modified nucleobase is a nucleobase that does not have a nucleobase, also referred to as an abasic nucleoside. In some embodiments, the abasic nucleoside is present in the internal portion of an oligonucleotide acceptor. In some embodiments, an abasic nucleoside is attached to the 3′- or 5′-terminal end, which is in certain embodiments grouped as a terminal group.

In some embodiments, the modified nucleobase is present on the 5′-terminal nucleoside of the oligonucleotide acceptor or oligonucleotide donor, 3′-terminal nucleoside of the oligonucleotide acceptor or oligonucleotide donor, and/or present on the internal nucleosides of the oligonucleotide acceptor or oligonucleotide donor. In some embodiments, the blocks or contiguous stretches of nucleosides in the oligonucleotide acceptor or oligonucleotide donor have modified nucleobases.

### Terminal Groups

In some embodiments, an oligonucleotide acceptor and/or nucleotide donor comprises a terminal group. In some embodiments, the oligonucleotide acceptor comprises a terminal group at the 5′-terminal nucleoside. In some embodiments, the terminal group is attached to the 5′-OH or 4′-carbon atom of the terminal nucleoside.

In some embodiments, the terminal group comprises a C-4′ modification of the 5′-terminal nucleoside, including among others, 4′-thio-C2′ modifications, 4′-aminoalkyl, C4′-guanidino-C2′-modifications, and C4′-O-methyl (see, e.g., Gangopadhyay et al., RNA Biology, 2022, 19:1, 452-467)

In some embodiments, the 5′-terminal group is a 5′-phosphate modification. In some embodiments, the 5′-phosphate modification, includes, among others, 5′-C-methyl, particularly S isomer; 5′-(E or Z)-vinylphosphonate, or 5′-methylenephosphonate.

In some embodiments, the 5′-terminal group comprises an abasic nucleotide attached to the 5′-OH. In some embodiments, the 5′-terminal groups comprises an inverted abasic nucleotide (5′-5′) attached to the 5′-OH of the 5′-end nucleoside.

In some embodiments, the nucleotide donor comprises a 3′-terminal group. In some embodiments, the 3′-terminal group comprises a 3′-phosphate, which can also function as a reversible blocking group. In some embodiments, the 3′-phosphate is modified, such as with 3′-(E or Z)-vinylphosphonate, or 3′-methylenephosphonate. In some embodiments, the 3′-terminal group on the nucleotide donor comprises an abasic nucleoside. In some embodiments, the 3′-terminal group comprises an inverted abasic nucleotide (3′-3′).

### Internucleoside Linkages

In some embodiments, the modified oligonucleotide comprises at least one modified, non-naturally occurring internucleoside linkage. In some embodiments, the modified oligonucleotide has 1%, 2%, 5%, 10% 20%, 30%, 40%, 50%, or 60% or more modified internucleoside linkages. In some embodiments, all of the internucleoside linkages are modified internucleoside linkages.

In some embodiments, the modified internucleoside linkage is a phosphorous containing modified internucleoside linkage. Exemplary phosphorous-containing internucleoside linkages include, among others, phosphotriesters, alkylphosphonates (e.g., methyl phosphonate, ethyl phosphonate, etc.), phosphoramidates, phosphorothioate, and phosphorodithioate.

In some embodiments, the modified internucleoside linkage is a non-phosphorous containing internucleoside linkage. Exemplary non-phosphorous containing internucleoside linkages include, among others, methylenemethylimino (—CH₂—N(CH₃)—O—CH₂), thiodiestcr, thionocarbamate (—O—C(═O)(NH)—S—); siloxane (—O—SiH₂—O—); N,N′-dimethylhydrazine (—CH₂—N((CH₃)—N((CH₃)—); MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). In some embodiments, the modified internucleoside linkage is amide linkage, such as those of glycine nucleosides or nucleoside β-amino acids (see, e.g., Banerjee et al., Bioconjugate Chem., 2015, 26, 8, 1737-1742).

In some embodiments, the modified internucleoside linkages provides for a chiral center. For example, a phosphorothioate or alkylphosphonate internucleoside linkage can be in the Rp or Sp stereometric configuration. In some embodiments, the oligonucleotide has a mixture of stereoisomers in the internucleoside linkage. In some embodiments, the oligonucleotide has greater than 50% of the internucleoside linkages as Rp or Sp configuration. In some embodiments, the oligonucleotide has at least 60%, 70%, 80%, 90%, or greater of Rp or Sp stereometric configuration.

In some embodiments, the modified internucleoside linkages are present in the 5′-terminal region of the oligonucleotide. In some embodiments, at least 1, 2, 3, 4, or 5 modified internucleoside linkages are present at the 5′-terminal region of the oligonucleotide. In some embodiments at least 1 or 2 phosphorothioate internucleoside linkages are present at the 5′-terminal region of the oligonucleotide. In some embodiments, the phosphorothioate linkage is a non-bridging phosphorothioate internucleoside linkage.

In some embodiments, the modified internucleoside linkages are present in the 3′-terminal region of the oligonucleotide. In some embodiments, at least 1, 2, 3, 4, or 5 modified internucleoside linkages are present at the 3′-terminal region of the oligonucleotide. In some embodiments, at least 1 or 2 phosphorothioate internucleoside linkages are present at the 3′-terminal region of an oligonucleotide.

In some embodiments, the modified internucleoside linkages are present in the internal portions of the oligonucleotide.

In some embodiments, the oligonucleotide comprises at least a phosphorothioate internucleoside linkage, where the phosphorothioate linkage is in the Sp configuration, the Rp configuration, or a mixture of Sp and Rp configurations in a population of the oligonucleotides.

### Conjugate Moiety

In some embodiments, the oligonucleotide comprises a conjugate moiety. In some embodiments, the nucleotide donor comprises a conjugate moiety that is compatible with single-stranded RNA ligase activity and that does not interfere with the nucleotide donor acting as a substrate for the single-stranded RNA ligase.

In some embodiments, the conjugate moiety (i.e., non-nucleotide moiety) includes, among others, carbohydrates (e.g. GalNAc), lipids, sterols, drug substances, hormones, polymers (e.g., polyethylene glycol, etc.), proteins, peptides, toxins (e.g. bacterial toxins, etc.), vitamins (e.g., folate, tocopherol, retinoic acid, etc.), or combinations thereof. In some embodiments, the conjugate moiety is used to affect the pharmacokinetics of the oligonucleotide and/or oligonucleotide cell targeting.

In some embodiments, the conjugate moiety can be attached to the 5′-terminal nucleotide, the 3′-terminal nucleotide, or in an oligonucleotide an internal nucleotide. In some embodiments, the conjugate moiety is attached the 2′-position of the sugar moiety of a nucleoside, for example, to the 2′-OH. In some embodiments, the conjugate moiety is attached to the 3′-position of the sugar moiety of the nucleoside, for example 3′-OH. In some embodiments, the conjugate moiety is attached to the nucleobase, as discussed above (see, e.g., Biscans et al., Nucleic Acids Res. 2019 Feb. 20; 47(3): 1082-1096). In some embodiments, the conjugate moiety is attached directly or attached using a linker.

In some embodiments, the conjugate moiety comprises a C₆-C₂₂ alkyl, C₆₋₂₂ alkenyl, or C₆-C₂₂ alkynyl. In some embodiments, the conjugate moiety comprises a C₆-alkyl, C₇-alkyl, C₈-alkyl, C₉-alkyl, C₁₀-alkyl, C₁₁-alkyl, C₁₂-alkyl, C₁₃-alkyl, C₁₄-alkyl, Cis-alkyl, C₁₆-alkyl, C₁₇-alkyl, Cis-alkyl, C₁₉-alkyl, C₂₀-alkyl, C₂₁-alkyl, or C₂₂-alkyl. In some embodiments, the conjugate moiety comprises a C₆ alkenyl, C₇ alkenyl, C₈ alkenyl C₉ alkenyl, C₁₀ alkenyl, C₁₁-alkenyl, C₁₂-alkenyl, C₁₃-alkenyl, C₁₄-alkenyl, Cis-alkenyl, C₁₆-alkenyl, C₁₇-alkenyl, C₁₈-alkenyl, C₁₉-alkenyl, C₂₀-alkenyl, C₂₁-alkenyl, or C₂₂-alkenyl. In some embodiments, the conjugate moiety comprises a C₆ alkynyl, C₇ alkynyl, C₈ alkynyl, C₉ alkynyl, C₁₀ alkynyl, C₁₁-alkynyl, C₁₂-alkynyl, C₁₃-alkynyl, C₁₄-alkynyl, C₁₅-alkynyl, C₁₆-alkynyl, C₁₇-alkynyl, C₁₈-alkynyl, C₁₉-alkynyl, C₂₀-alkynyl, C₂₁-alkynyl, or C₂₂-alkynyl.

In some embodiments, the conjugate moiety comprises a heteroalkyl, heteroalkenyl, or heteroalkynyl. In some embodiments, the heteroalkyl, heteroalkenyl or heteroalkynyl has one or more carbon atoms replaced with a heteroatom, such as O, S, or N.

In some embodiments, the conjugate moiety comprises a cycloalkyl or heterocycloalkyl group. In some embodiments, the cycloalkyl includes, among others, cyclopropyl, cyclobutyl, cyclopentyl, cyclohexyl, 1-cyclohexenyl, 3-cyclohexenyl, and cycloheptyl. In some embodiments, the heterocycloalkyl includes, among others, 1-(1,2,5,6-tetrahydropyridyfh 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, and 2-piperazinyl.

In some embodiments, the conjugate moiety comprises an aryl or heteroaryl moiety. In some embodiments, the aryl group includes, among others, phenyl, naphthyl, indenyl, biphenyl, phenanthrenyl, naphthacenyl, anthracenyl, fluorenyl, indenyl, and azulenyl. In some embodiments, a heteroaryl group includes, among others, pyridyl, furanyl, thienyl, pynolyl, oxazolyl, oxadiazolyl, imidazolyl ihiazolyl, isoxazolyl, quinolinyl, pyrazolyl, isoihiazolyl, pyridazinyl, pyrimidinyl, pyrazinyl, triazinyl, isoquinolinyl, and indazolyl.

In some embodiments, the conjugate moiety comprises a cycloalkylalkyl-, heterocycloalkylalkyl-, arylalkyl-, heteroarylalkyl-, cycloalkylheteroalkyl-heterocycloalkylheteroalkyl-, arylheteroalkyl-, heteroarylheteroalkyl-, cycloalkylalkenyl-, heterocycloalkylalkenyl-, arylalkenyl-, heteroarylalkenyl-, cycloalkylheteroalkenyl-heterocycloalkylheteroalkenyl-, arylheteroalkenyl-, or heteroarylheteroalkenyl-.

In some embodiments, the conjugate moiety comprises a lipid or lipophilic moiety, for example a fatty acid. In some embodiments, the fatty acid comprises a saturated fatty acid, unsaturated fatty acid, or a polyunsaturated fatty acid. In some embodiments, the fatty acid comprises caprylic acid, lauric acid, myristic acid, palmitic acid, stearic acid, arachidic acid, behenic acid, oleic acid, elaidic acid, cis-vaccenic acid, trans-vaccenic acid, linoleic acid, alpha-linoleic acid, gamma-linoleic acid, arachidonic acid, eicosapentaenoic acid, decanoic acid, docosahexaenoic acid (DHA), and docosanoic acid (DCA) conjugate moieties (see, e.g., Kubo et al., ACS Chem. Biol., 2021, 16, 150-164; see also, WO2024/040041; incorporated herein by reference).

In some embodiments, the conjugate moiety comprises a sterol. In some embodiments, the sterol comprises cholesterol, alpha-cholesterol, cholesterol ester (e.g., cholesteryl palmitate, etc.), cholesterol sulfate, phytosterol, cholic acid, or lithocholic acid.

In some embodiments, the conjugate moiety comprises a phospholipid. In some embodiments, the phospholipid comprises phosphatidic acid, phosphatidylethanolamine, phosphatidylcholine, phosphatidylinositol, phosphatidylserine, or a sphingolipid.

In some embodiments, the conjugate moiety comprises a carbohydrate, particularly a carbohydrate moiety acting as a ligand for a cellular receptor for cellular targeting of the oligonucleotide. In some embodiments, the carbohydrate moiety comprises galactose or galactose derivatives. In some embodiments, the carbohydrate moiety is attached to the nucleoside via a linker. In some embodiments, exemplary carbohydrates that can be used include the following.

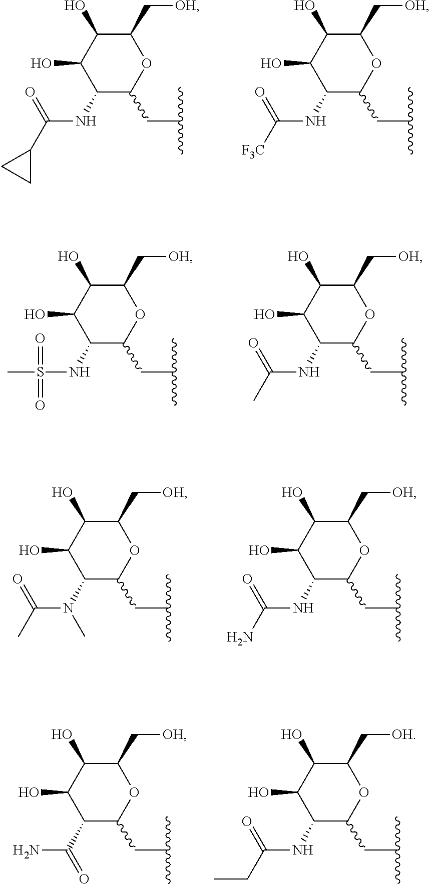

In some embodiments, the conjugate moiety is an N-acetylgalactosamine (GalNAc) conjugate moiety. In some embodiments, the oligonucleotide may be conjugated to at least one conjugate moiety comprising at least one N-acetylgalactosamine (GalNAc) moiety. In some embodiments, the conjugate moiety is a monovalent, divalent, trivalent or tetravalent, GalNAc.

In some embodiments, the GalNAc moiety has the following structure,

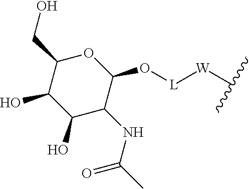

where L is a linker, and W is a heteroatom (e.g., O or N). In some embodiments, the W is the 2′-OH of the sugar moiety of a nucleoside. In some embodiments, the L includes a 3′-phosphate at the 3′-terminal nucleoside. An exemplary monovalent GalNAc moiety is

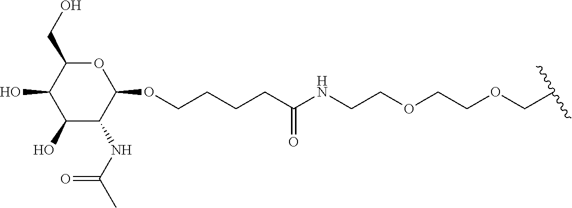 [SMILES: *COCCOCCNC(=O)CCCCO[C@@H]1O[C@H](CO)[C@H](O)[C@H](O)[C@H]1NC(C)=O]

wherein the monovalent GalNAc is attached via the linker to the 2′-position of a nucleoside, such as adenine or guanine. These conjugate moieties can be present in contiguous nucleotides in an oligonucleotide (see, e.g., WO2024/040041).

In some embodiments, the conjugate moiety is a trivalent GalNAc. Tri-valent N-acetylgalactosamine conjugate moieties are described in, for example, WO 2014/076196, WO 2014/207232 and WO 2014/179620. The term “trivalent GalNAc” refers to a residue comprising three N-acetylgalactosamine moieties, typically attached via a linker. Exemplary trivalent GalNAc conjugate moiety is depicted below:

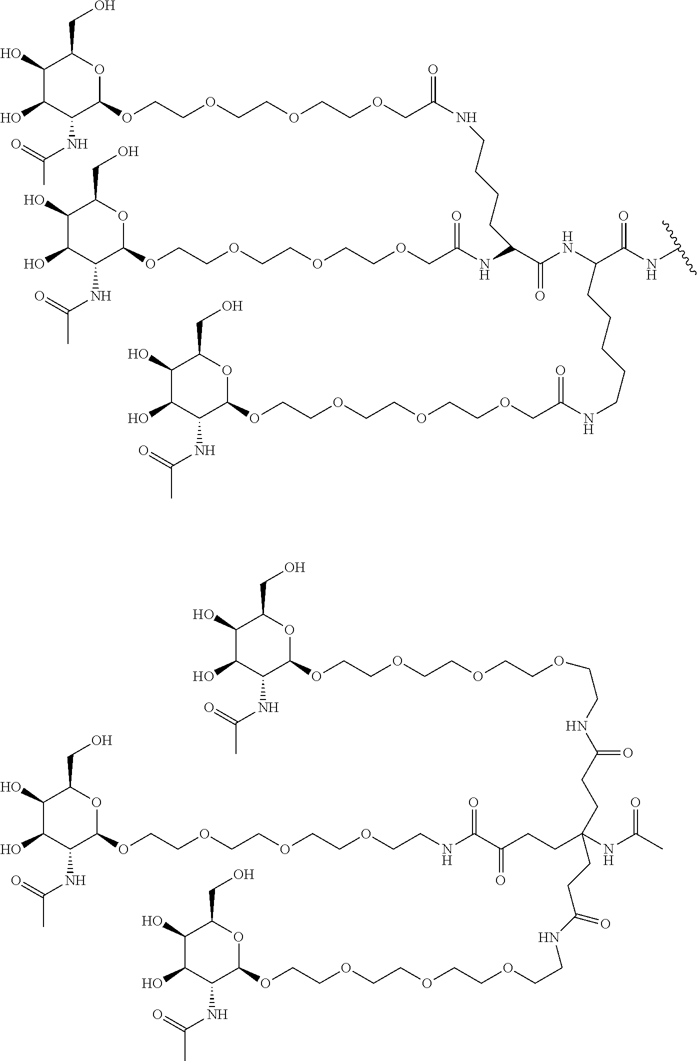

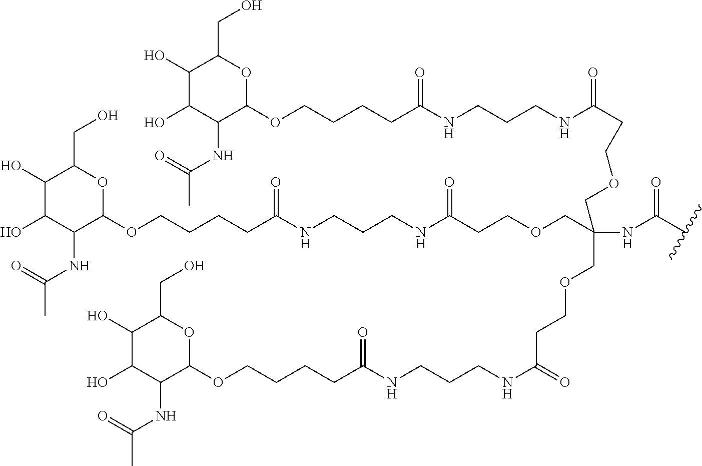 [SMILES: *C(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some embodiments, the conjugate is GalNAc targeting moiety L96.

In some embodiments, the conjugate moiety comprises a reporter molecule. Examples of reporter molecules include, among others, fluorescent moieties, such as fluorescein and fluorescein dyes (e.g., fluorescein isothiocyanine or FITC, naphthofluorescein, 4′,5′-dichloro-2′,7′-dimethoxy-fluorescein, 6-carboxyfluorescein or FAM), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g., Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800). (See, e.g., “The Handbook of Fluorescent Probes and Research Products”, 9th Ed., R. P. Haugland, 2002, Molecular Probes, Inc., Eugene, Oregon)

In some embodiments, the reporter moiety is a chemiluminescent moiety, for example acridinium esters, ruthenium derivatives (e.g., tris(2,2′-bipyridyl) ruthenium), and dioxetanes.

In some embodiments, the conjugate moiety comprises an affinity or capture tag. Exemplary affinity or capture tag includes, among others, biotin, desthiobiotin, digoxigenin, 3-amino-3-deoxydigoxigenin, and a hapten (e.g., dinitrophenol, Alexa Fluor 40, Alexa Fluor 488, dansyl, Lucifer yellow, Oregon Green 488, fluorescein).

In some embodiments, the conjugate moiety comprises a peptide. In some embodiments, the peptide comprises a cellular targeting peptide and/or cell penetration peptide (CPP) for enhancing cellular delivery of a conjugate modified oligonucleotide. In some embodiments, the cell penetrating peptide is attached via a linker, including a cleavable linker. Cell penetrating peptides, include among others, TAT, penetratin, MAP, transportan/TP10, VP22, polyarginine, MPG, Pep-1, pVEC, YTA2, YTA4, M918, and CADY. In some embodiments, the conjugate moiety comprises an RGD (Arg-Gly-Asp) peptide. Sequence of some penetrating peptides are described in Copolovici et al., 2014, 8(3):1972-1994 and some are provided below:

| CPP Peptide | Peptide Sequence |
|---|---|
| TAT | GRKKRRQRRRPPQ |
| penetratin | RQIKIWFQNRRMKWKK |
| MAP | KLALKLALKALKAALKLA |
| Transportan/TP10 | GWTLNS/AGYLLGKINLKALAALAKKIL |
| Transsortan9/TP9 | LKALLGKIN |
| VP22 | NAKTRRHERRRKLAIER |
| Polyarginine | (R)n, where n is 8 or 9 |
| MPG | GALFLGFLGAAGSTMGA |
| Pep-1 | KETWWETWWTEWSQPKKKRKV |
| pVEC | LLIILRRRIRKQAHAHSK |
| YTA2, | YTAIAWVKAFIRKLRK |
| YTA4, | IAWVKAFIRKLRKGPLG |
| M918 | MVTVLFRRLRIRRACGPPRVRV |
| CADY | GLWRALWRLLRSLWRLLWRA |
| RGD | RGD |

Other cell penetrating peptides, including those conjugated to nucleic acids, are disclosed in, among others, patent publications WO24063570, WO24044663, US2024083949, WO24026141, WO23230600, WO23219933, WO23177261, WO23178327, WO23093960, WO23086342, WO23081893, WO23069332, WO23070108, WO23034515, US2023248630, US2023053924, WO23003380, WO23277628, WO23277575, US2022378946, WO22171972, WO22162200, WO2020144233, WO22180242, WO22132520, WO22129926, WO22125673, WO22120276, WO22101193, US2023287086, US2023357334, US2023144488, and US2023048338; incorporated by reference herein. In some embodiments, the peptide can be attached using a thiol group on the 5′-phosphate of a polynucleotide or oligonucleotide.

Exemplary nucleotides/nucleosides with conjugate moieties are shown below:

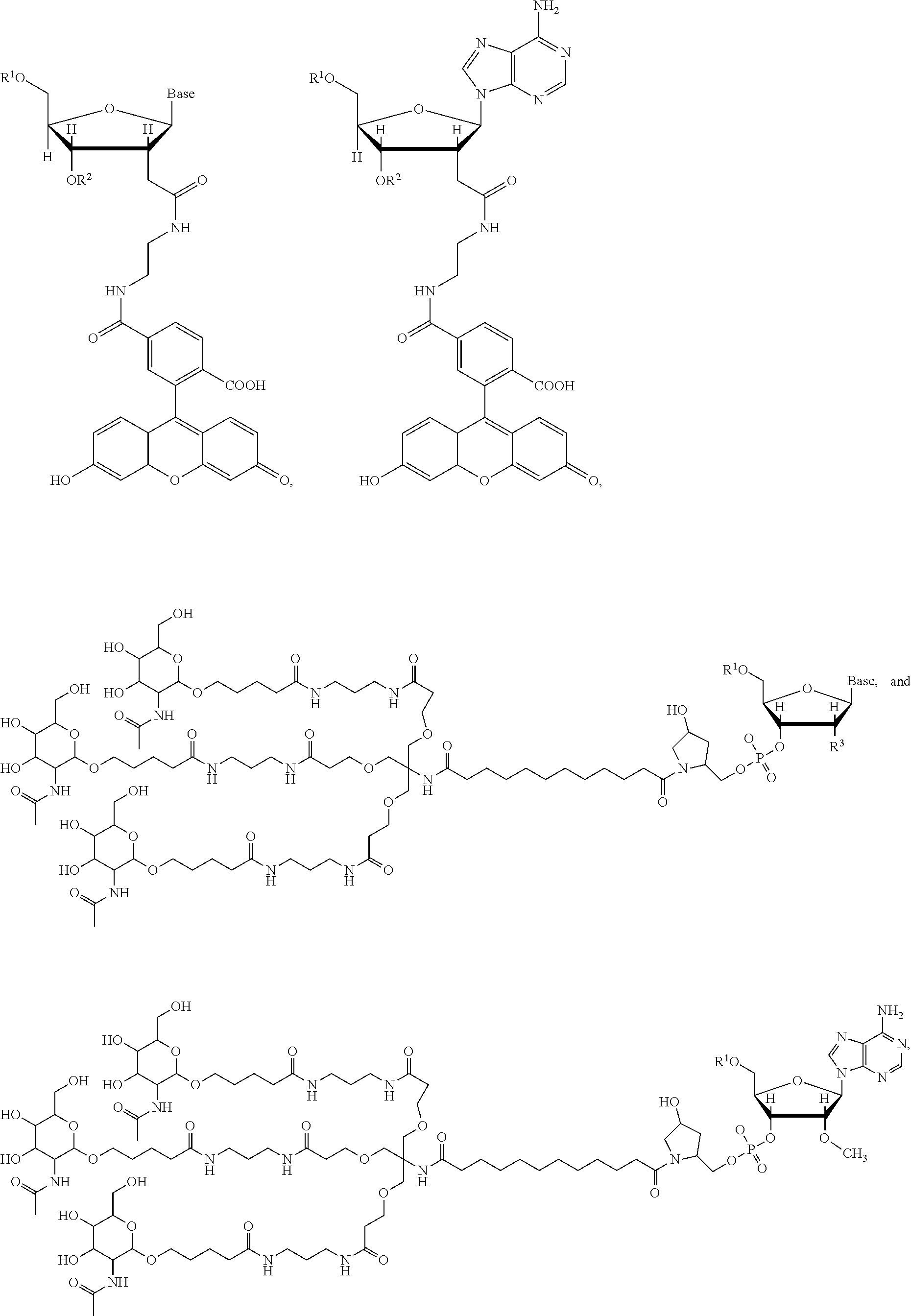

wherein R¹ is H or phosphate group, R² is H or phosphate group, or blocking group, and R³ is H, OH, fluoro, or —O-methyl.

### Reactive Moiety

In some embodiments, the modification comprises a reactive group that is conjugated to a nucleoside. In some embodiments, the reactive group is attached to the nucleoside via a linker. In some embodiments, the reactive group is a cyano, azido, alkynyl, amino, carboxyl, sulfhydryl, dibenzocyclooctynyl, vinyl, trans-cyclooctene, or tetrazine. In some embodiments, the reactive group is those used for click chemistry, including copper free click chemistry. Exemplary reactive groups are provided below:

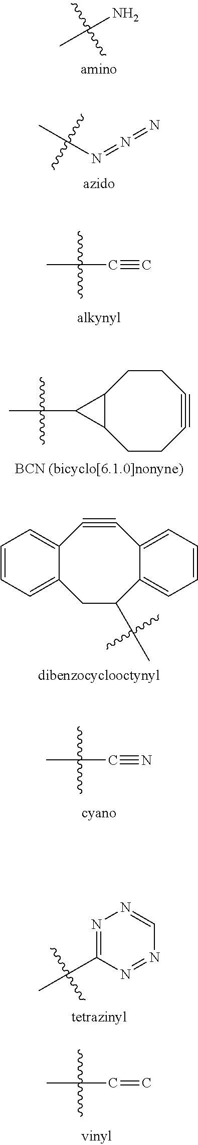

Other reactive groups used in click chemistry, particularly for nucleic acids, is described in Fantoni et al., Chem. Rev. 2021, 121, 7122-7154, incorporated by reference herein.

Exemplary single stranded RNA ligase substrates with a reactive moiety is provided below:

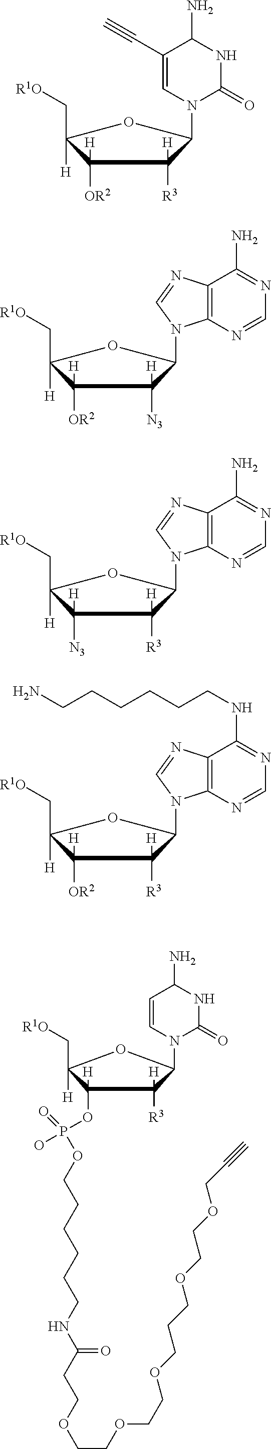

wherein R¹ is H or phosphate; R² is a blocking group or H; and R³ is H, —OR, or halo, e.g., F, Br, or Cl.

### Linker

In some embodiments, as described above, the conjugate moiety or reactive moiety is attached to the nucleoside or the terminal group through a linker. Various linkers are known in the art for conjugating chemical groups to nucleosides and phosphate groups.

In some embodiments, mixtures of linkers are used. In some embodiments, different linker types are connected to form a longer linker or linkers with branched or dendritic structure. For example, an alkylene linker is connected to a polyethylene linker through a functional group, e.g., an amide; an arylene linker is attached to an alkylene linker. As such, different combinations of linker types can be connected to provide for longer linkers and/or branched linkers, for example for attaching multiple conjugate moieties.

In some embodiments, linkers include, among others, substituted or unsubstituted alkylene, heteroalkylene, alkenylene, heteroalkenylene, arylene, heteroarylene, arylalkylene, arylalkenylene, heteroarylalkylene, heteroarylalkenylene, arylheteroalkylene, arylheteroalkenylene, heteroarylheteroalkylene, and heteroarylalkenylene. In some embodiments, the linker comprises substituted or unsubstituted C₂-C₂₂ alkylene, heteroalkylene, or polyethylene glycol. In some embodiments, the linkers have functional groups for conjugation.

In some embodiments, the linker comprises a divalent, branched or unbranched, saturated or unsaturated, hydrocarbon chain, having from 1 to 50 carbon atoms, 1 to 20 carbon atoms, or 1 to 14 carbon atoms, wherein one or more of the carbon atoms in the hydrocarbon chain is optionally replaced by —O—, —NR¹—, —NR¹—C(═O)—, —C(═O)—NR¹, or —S—, and wherein R¹ is hydrogen or (C₁-C₆)alkyl, wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In some embodiments, the L is attached to the nucleoside and/or conjugate through —NH—, —O—, —S—, —(C═O)—, —(C═O)—NH—, —NH—(C═O)—, —(C═O)—O—, —NH—(C═O)—NH—, or —NH—(SO₂)—.

In some embodiments, the L has the structure below:

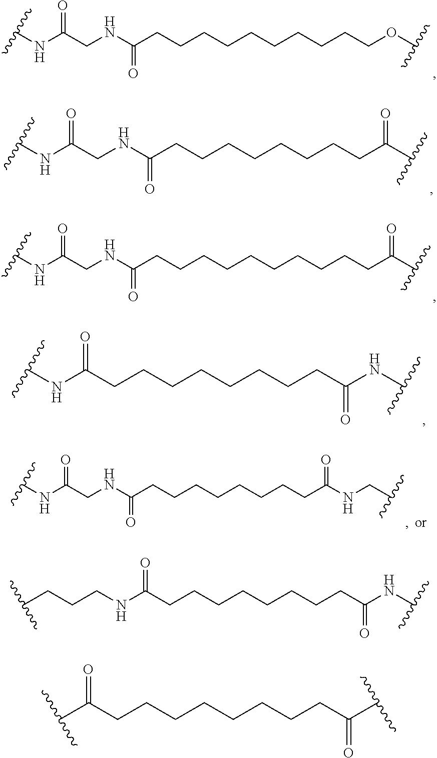

In some embodiments, the linker comprises a substituted or unsubstituted polyethylene glycol linker. In some embodiments, the polyethylene glycol linker has the formula:

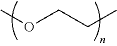

In some embodiments, n is 2-24. In some embodiments, n is 2, 3, 4, 5, 6, 7, 8, 9, 10, 11, 12, 13, 14, 15, 16, 17, 18, 19, 20, 21, 22, 23, or 24.

In some embodiments, the polyethylene linker has the structure below:

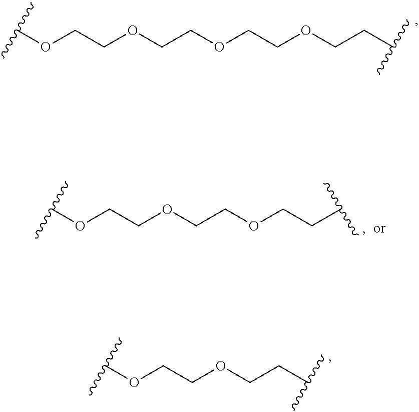 [SMILES: *CCOCCOCCOCCO* ; *CCOCCOCCO* ; *CCOCCO*]

In some embodiments, the polyethylene linker has the structure below:

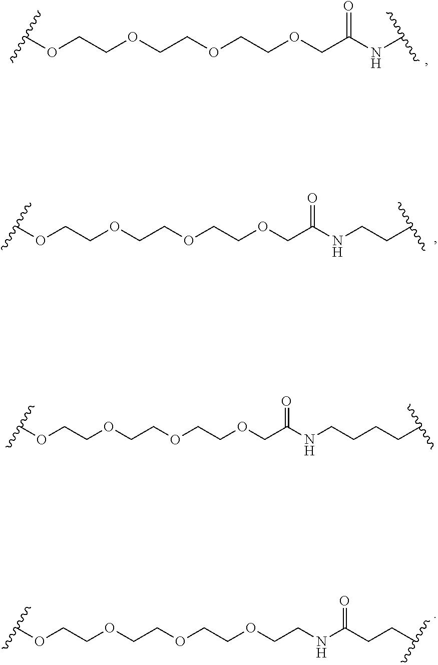 [SMILES: *NC(=O)COCCOCCOCCO* ; *CCNC(=O)COCCOCCOCCO* ; *CCCCNC(=O)COCCOCCOCCO* ; *CCC(=O)NCCOCCOCCOCCO*]

In some embodiments, the linker is a cleavable linker in which the linker can be cleaved, for example to detach a conjugate moiety. Example of a cleavable linker includes, by way of example and not limitation, a disulfide linkage, enzymatically cleavable linkers (e.g., peptide linkers), and photocleavable linkers (see, e.g., Hermanson, G., Bioconjugate Techniques, 3rd Ed., 2013, Academic Press; see also Bioconjugation Protocols: Strategies and Methods, In Methods in Molecular Biology, 2^{nd} Ed., S. S. Mark ed., 2011, Humana Press).

In some embodiments, bifunctional linkers can be used to attach a conjugate moiety to the linker and attach the linker-conjugate to the nucleoside or vice versa (see, e.g., Hermanson, G., supra; see also Bioconjugation Protocols: Strategies and Methods, In Methods in Molecular Biology, supra). In some embodiments, an activating group can be attached to an atom to activate the atom to form a covalent bond with another reactive group. Examples of synthetic activating groups that can be attached to an oxygen atom include, but are not limited to, acetate, succinate, triflate, and mesylate. When an activating group is attached to an oxygen atom of a carboxylic acid, the activating group can be a group that is derivable from a known coupling reagent. Examples of such coupling reagents include, but are not limited to, N,N′-dicyclohexylcarbodimide (DCC), hydroxybenzotriazole (HOBt), N-(3-dimethylaminopropyl)-N′-ethylcarbonate (EDC), (denzotriazol-1-yloxy)tris(dimethylamino)phosphonium hexafluorophosphate (BOP), benzotriazol-1-yl-oxytripyrrolidinophosphonium hexafluorophosphate (PyBOP) or O-benzotriazol-1-yl-N,N,N′,N′-tetramethyluronium hexafluorophosphate (HBTU).

### Determining Ligation Synthesis Strategies

As indicated, a trained machine learning model may use features of nucleic acid fragments to be ligated to predict one or more characteristics of a ligation reaction. Predicted characteristics of a ligation reaction may, in turn, be used recommend ligation synthesis strategies such as locations of ligation sites on a product molecule and/or choice of ligases.

The trained machine learning model may be integrated in an overall process flow that also employs feature extraction. In some implementations, a process flow begins by receiving structural information charactering a nucleic acid that is to be synthesized using a process that ligates oligonucleotide fragments. In some cases, the nucleic acid to be synthesized is a double stranded nucleic acid such as an siRNA molecule. The structural information characterizing the nucleic acid to be synthesized may comprise a nucleotide sequence of the double stranded nucleic acid and, optionally, one or more chemical modifications of the nucleotides of the double stranded nucleic acid. In some cases, the process flow constrains the structural information to only certain portions of the nucleic acid such only double stranded regions of a product molecule having both single and double stranded regions.

The process flow also identifies multiple possible ligation sites on the nucleic acid to be synthesized. Each of these sites represents a different ligation reaction that might be used in a synthesis scheme. The computational process evaluates these sites to help identify which ones are appropriate for the synthesis scheme. When the nucleic acid to be synthesized is a double stranded nucleic acid, the process may identify possible ligation sites on one or both strands of the double stranded nucleic acid.

After identifying the possible ligation sites, the computational process considers each such site, and for it identifies features characterizing the ligation site. In some implementations, the features comprise information about the types of nucleotides located at first defined positions with respect to the current ligation site and/or information about one or more chemical modifications of nucleotides located at second defined positions with respect to the current ligation site. The first and second defined positions may be chosen anywhere along the oligonucleotide fragments defined by the ligation site. In some cases, these positions are near the ligation site (e.g., within 10 nucleotides, or 8 nucleotides, or 6 nucleotides, or 5 nucleotides, or 4 nucleotides, or 3 nucleotides, or 2 nucleotides).

After the process identifies the features for a given ligation site, it employs a trained computational model to determine, based on the features for each of the ligation site, whether the ligation site meets a criterion for a ligation reaction to produce the double stranded nucleic acid. An example of such criterion is a predicted efficiency of the ligation reaction. In some cases, the trained computational model outputs a probability that the ligation reaction will have at least defined minimum efficiency. In some cases, the computation process outputs and ranks multiple combinations of ligation sites and associated ligases that meet the criterion for the ligation reactions at the combinations of ligation sites.

In some embodiments, computational method is constrained to not select a combination of ligation sites that will require a blunt end ligation. In some embodiments, computational method is constrained to select only combinations of ligation sites in which a single ligase can meet the criterion at all ligation sites. This ensures that the synthesis scheme need only employ a single ligation reaction. As an example, the computational process may be constrained to only select combinations of ligation sites in which all ligation sites in the combination are predicted to undergo a ligation reaction of at least a defined minimum efficiency when using the same ligase.

In some embodiments, the computational model is constrained to select only ligation sites that will result in oligonucleotide fragments having a particular size range such as about 5 to 30 nucleotides in length, or about 5 to 20 nucleotides in length.

Many different types of trained computational models may be employed. In some embodiments, the trained computational model comprises a regression model. In some embodiments, the trained computational model comprises a machine learning classification model. In some embodiments, the trained computational model comprises an ensemble of machine learning models.

Examples of suitable machine learning model types include classification models such as neural networks, support vector machines, decision tree-based models such as a random forest models, and gaussian process regression models. Other examples of suitable machine learning model types include regression models such as support vector regression models, partial least squares models, principal component regression models, and gaussian process regression models. Some examples of suitable machine learning model types include models trained with regularization including ridge models and lasso models. A representative list of models types include Ridge, Lasso, GPR (Gaussian Process Regression), PLS (Partial Least Squares), Random Forest, SVM (Support Vector Machine)

In some embodiments, a computation system is configured to receive as inputs features of a double stranded RNA product (e.g., siRNA) to be synthesized by ligation. The input features include sequence information, chemical modifications of any nucleotides (fluorination, methylation, etc.), and the like. The computational system is configured to output ligation sites, and optionally associated oligonucleotide fragments of the RNA product, a double stranded RNA ligase choice; and predicted efficiency of ligation reaction.

In some embodiments, a sequence of operations for using the computational system include:
Receiving as input a nucleic acid sequence of a nucleic acid product including any chemical modification to nucleotides;
Featurization of input nucleic acid sequence;
Prediction of ligation efficiency for each of multiple possible ligation sites on the nucleic acid sequence;
Outputting locations of one or more ligation sites on one or both strands of the nucleic acid product, and probabilities that an efficient ligation will take place at the ligation sites using the chosen ligase.

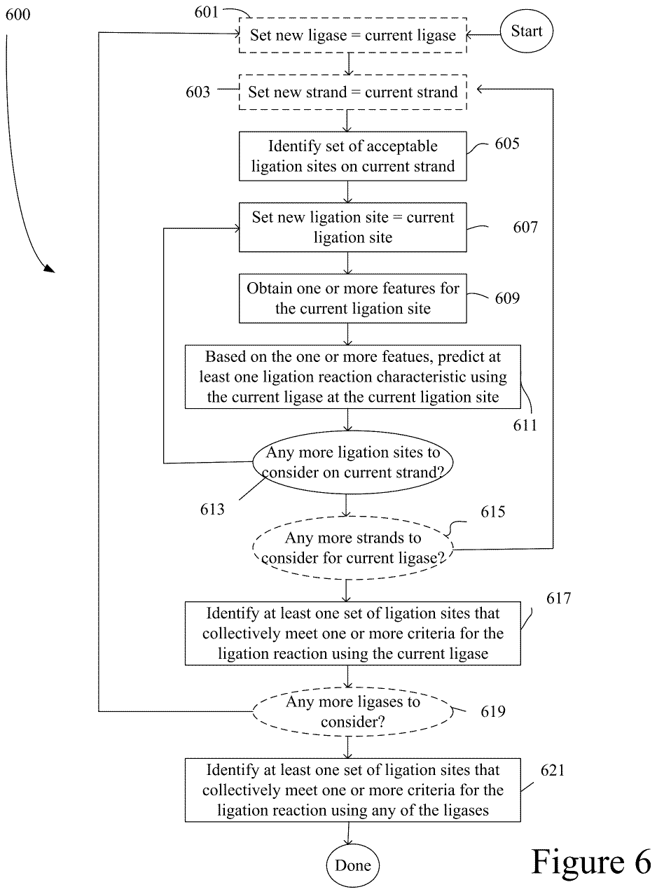

FIG. 6 presents one example of a computation process 600 for determining a ligation-based synthesis strategy for producing a nucleic acid such as a double stranded nucleic acid. A computational system is employed to execute the computational process. The computation system may employ a machine learning model and a feature extractor to perform certain parts of the computational process. Aspects of suitable models and feature extractors are presented elsewhere herein.

FIG. 6 may be viewed as implementing the inference phase a machine learning process in which a training phase trains the machine learning model, and the inference phase applies the trained machine learning model to predict or suggest an outcome.

As illustrated, process 600 identifies suitable ligation sites for a synthesis scheme that produces the double-stranded nucleic acid. The depicted embodiment employs three nested algorithmic loops: a first or inner most loop that iterates over ligation sites on a strand of a nucleic acid, a second loop that iterates over individual strands of a double-stranded nucleic acid, and a third or outer loop that iterates over multiple ligases under consideration. It should be understood that there are many variations of the depicted embodiment, including some that consider only a single strand and/or a single ligase. In such cases or more of the loops in the depicted embodiment are not present.

As illustrated, the computational process begins at an optional operation 601 by selecting a ligase to evaluate for potential use in a ligation scheme. This operation is optional because in some embodiments, only one ligase will be considered.

After the current (or only) ligase is set at operation 601, the process considers a number of potential sites on the nucleic acid for ligation reactions involving the currently selected ligase. In some but not all embodiments, multiple ligases are considered in this way sequentially.

After operation 601 is optionally performed, the computational process optionally selects a current strand of the nucleic acid to analyze. See operation 603. In the case of a double stranded nucleic acid, there will be only two strands to consider, but this is not always the case. In some examples, a single-stranded nucleic acid is considered. In other cases, a nucleic acid having three or more strands is considered.

After the current strand is set in operation 603, the process identifies a set of acceptable ligation sites on the current strand. See operation 605. While the sequence of a given stand has a number of potential ligation sites equal to N−1, where N is the number of nucleotides in a strand, there may be certain constraints in choosing ligation sites. For example, if the range of acceptable strand lengths is between x and y nucleotides, a ligation site cannot be closer to a terminus of the strand than a distance of x nucleotides. Similarly, the ligation site cannot result in a fragment having a length of greater than y nucleotides. Additionally, in some embodiments, the computational method excludes ligation sites that would result in a blunt end double stranded ligation.

With appropriate ligation sites identified in operation 605, the computational process can iterate over those sites as indicated in a loop defined by operations 607, 609, 611, and 613. In the first of these operations, operation 607, the computational method chooses a current ligation site in the current strand. Then, at an operation 609, the computational method obtains a set of features for the current ligation site. This operation may be implemented by a computational module sometimes referred to as a feature extractor.

As explained elsewhere herein, the features obtained for any given ligation site can include a number of pieces of information such as nucleotide sequence, proximity to other ligation sites (e.g., on a different strand), chemical modifications to nucleotides in the nucleic acid, and the like. Further, with respect to the ligation site, the features may be found within a bounded region or window such as within a certain number of nucleotides upstream and/or downstream from the ligation site.

After obtaining the features at the current ligation site, the computational system uses the feature information to predict one or more characteristics of a ligation reaction at the ligation site using the ligase currently under consideration. See operation 611, which may be implemented using a trained machine learning model. As explained, one example of a characteristic of the ligation reaction is a probability that the reaction will ligate two fragments at a ligation site with at least a defined minimum value of coupling efficiency, e.g., a probability that the reaction at the current site will occur with greater than 90% coupling efficiency.

It should be understood that while operation 611 predicts a ligation reaction characteristic for a particular ligation site, which is the current ligation site, the predicted ligation characteristic may depend on the positions or other features of one or more other ligation sites, which may have been fixed earlier in the process. For example, if a first ligation site has been fixed, one or more features of the current ligation site may depend on the location or other information about the first ligation site. For example, a separation distance (optionally in nucleotides) between current ligation site and the first ligation site may be a feature used by the machine learning model to predict the reaction characteristic at the current ligation site.

In addition to predicting such characteristic or characteristics, the computational system may also determine whether the characteristic meets a particular criterion for the ligation reaction to be deemed successful, or at least a candidate for a desired ligation-based synthesis scheme. For example, the computation method may require that a ligation reaction have at least a 90% probability that the reaction at the current site will occur with greater than 90% coupling efficiency.

Note however that whether a ligase and a set of ligation sites is selected for a reaction scheme ultimately depends on how well the ligation reaction is predicted to proceed at each of the multiple ligation sites in a combination of such sites. In some embodiments, the predicted characteristic of a ligation reaction at the current site is simply stored for later consideration when determining an appropriate combination of ligation sites for use with the current ligase.

After operation 611 is completed, the predictive machine learning model next determines whether any more ligation sites remain to be considered on the current strand. See decision operation 613. If the method determines that more ligation sites remain to be considered, process control returns to operation 607 where the next ligation site on the current strand is selected for further evaluation. Thereafter, operations 609, 611, and 613 execute for the new current site. Note that the new site will typically have a different set of feature values than the previous site. Thus, the characteristics of the ligation reaction, as predicted at operation 611, will vary from ligation site to ligation site.

After the computational method determines that there are no more ligation sites to consider on the current strand, a loop control operation 615 determines whether any additional strands remain to be analyzed. If one or more such strands remain, process control is directed back to operation 603 which sets a new current strand for evaluation in accordance with the description of operations 605, 607, 609, 611, 613, and 615.

When operation 615 determines that no other strands remain to be analyzed, an operation 617 attempts to identify a set of ligation sites that meet a criterion for the ligation reaction using the current ligase. For example, as mentioned, in order for a ligation site to be considered in a synthesis scheme for double-stranded nucleic acid under consideration, it may have to have a high probability of a high coupling efficiency. Note also that operation 617 may identify multiple combinations of ligation sites and optionally rank them.

In some embodiments, in order for a set of ligation sites to be deemed acceptable, each site must meet the above-mentioned criterion for a successful ligation reaction. In some embodiments, however, the sites collectively must meet a criterion. For example, values of the reaction characteristic determined at operation 611 for the individual sites of a potential set of sites may be averaged or otherwise considered together to determine whether the potential set of sites is acceptable. Further predictions of a reaction characteristic for any given ligation site may be dependent on the location or locations of one or more other ligation sites, optionally on different strands. As indicated, some of the predictions made at operation 611 may assume that the current ligation position is some distance away from a different ligation site, which may be fixed. So, the choice of whether to include any particular ligation site in a combination of ligation sites as identified in operation 617 may, in some cases, not be conducted in isolation, but rather be conducted with respect to one or more other ligation sites for inclusion in the combination of ligation sites.

Further, operation 617 may save the results for later comparison against characteristics of reactions involving one or more different ligases at different combinations of ligation sites.

After potentially identifying and outputting one or more sets of ligation sites for the current ligase, the computational method determines whether there are any more ligases to consider. See decision operation 619. If additional ligases should be considered, process control returns to operation 601, where the next ligase to be considered is set to the current ligase, and the process of analyzing multiple ligation sites for the newly current ligase proceeds as described above. In some cases, only a single ligase is considered and operation 619 need not be performed. In some cases, separate machine learning models to predict characteristics of ligation reactions using different ligases. For example, a first model may be trained for a first ligase, a second model may be trained for a second ligase, etc.

After all ligases have been considered, the computation process may evaluate the various combinations of ligation sites for the various ligases and determine which of them provides the best chance for a successful synthesis scheme. See operation 621. The process may also output one or more such ligation schemes for the one or more ligases. The process may rank such schemes for likelihood of providing an acceptable ligation strategy for synthesizing the double-stranded nucleic acid.

### Training Machine Learning Models to Predict Ligation Reaction Characteristics

Machine learning models described herein may be trained using any suitable training algorithm including off the shelf algorithms. Some such techniques employ supervised or semi-supervised training.

In certain embodiments, a machine learning model is trained using a cost function such as a mean squared error (MSE) function for multivariate training data. This approach uses a sum of weighted error between the model prediction and actual labels of the training data over multiple dimensions. The multivariate information may include multiple pieces of information about ligation reactions including the identify of the ligases, features of the ligation sites, and characteristics of the ligation reactions (labels).

### Ligation Reaction Data

For each product nucleic acid used to provide training data (e.g., siRNA molecules), prepare multiple nucleic acid ligation substrates, each having one or more nicks at defined locations (ligation sites).

For each ligation substrate having one more nicks at defined locations, perform one or more ligation reactions and determine one or more characteristics for the ligation reaction(s). For example, determine the percent conversion for each ligation reaction. For each ligation substrate, optionally perform multiple ligation reactions, each with a different ligase.

The ligation reaction characteristic(s) may serve as labels in supervised or semi-supervised training.

### Featurization

For each product nucleic acid used to provide training data, feature data is obtained. An example of how to receive such data follows:
Receive sequence of product nucleic acid molecule to synthesized.
Optionally remove regions that will not be relevant to prediction. E.g., in a double stranded product nucleic acid, remove single stranded region(s).
Code for modified nucleotides. E.g., 2-methyl or 2-fluoro modifications may be encoded with binary encoding.
Code for sequence. E.g., encode bases A (adenine), C (cytosine), G (guanine), U (uridine) as such. E.g., encode as “structure” and “base pairing”. A and G have the same structure value (0 or 1) as they are both purines. C and U are both pyrimidines and have the opposite value than A and G. A and U share a base pairing value, as do C and G.
Code location of ligation sites. E.g., encode the ligation site positions as the distances from the 5′ end of a given strand, the distances from the 3′ end of the strand, and/or as the size of the shortest RNA fragment (the minimum of the 2 prior features).
Code the positions of two ligation sites relative to each other. These may be represented as two features: the distance between the sites in terms of number of bases and the “sign”, which represents whether the other site is on the 3′ side (1) or the 5′ side (0) of its own strand.

### Training a Model

For each ligation site, select a site-relevant subset of the feature values and a measured characteristic of the ligation reaction for the ligation site. Examples of site-relevant subsets of the feature values include sequence and chemical modification information for regions of sequence centered from the ligation site such as [−2, +2], [−6, +5], and [−3, +3], along with the corresponding opposing strand sequences and modifications. The combinations of ligation site, site-relevant feature values, and characteristic of the ligation reaction comprise data points of the training data.

Provide the training data to an appropriate model training routine. Follow with cross validation and evaluation. In certain embodiments, model being trained may be evaluated in regression and/or binary classification modes. Models may be trained for a ligase individually or for multiple ligases. Models may be built in any suitable coding language such as python and utilizing publicly available packages such as sklearn.

Supervised models generated as described allowed selection of the high performing combinations of ligases and oligonucleotide fragment designs. Top models achieved greater than 85% classification accuracy and 100% precision when tested on novel siRNA designs, significantly improving the identification of top designs over random screening. The use of ML reduces the need for additional screening, accelerates workflows and provides insight into further improvements.

### Ligases

The disclosed methods and models for predicting ligation reaction characteristics may be used for any of many types of ligases. Such ligases may act on single stranded nucleic acids, double stranded nucleic acids, RNA ligases, DNA ligases, etc. Further the ligases may be naturally occurring, wild type ligases or engineered ligases.

Examples of RNA ligases for which reaction characteristics may be predicted are described in PCT Patent Application No. PCT/US2025/010294, filed Jan. 3, 2025, which is incorporated herein by reference in its entirety.

### Ligation Reactions

In some embodiments, the method includes a nucleotide cofactor or substrate used by the RNA ligase to catalyze the joining reaction. In some embodiments, the nucleotide cofactor or substrate is ATP and/or dATP. Preferably, the nucleotide cofactor or substrate is ATP. In some embodiments, the reaction conditions for the ligation include additional components, such as Mg⁺², buffer, and/or salts.

In some embodiments, the nucleotide cofactor or substrate (e.g., ATP) is present at a concentration of about 0.05-25 mM, 1-20 mM, 2-18 mM, or 5-15 mM. In some embodiments, the nucleotide cofactor or substrate is present at a concentration of about 0.5 mM, 1 mM, 2 mM, 3 mM 4 mM, 5 mM, 6 mM, 7 mM, 8 mM, 9 mM, 10 mM, 12 mM, 15 mM, 20 mM, or 25 mM.

In some embodiments, the reaction conditions also include a ligation enhancing reagent, including, among others, DMSO, betaine, polyethylene glycol (e.g., PEG 6000, PEG 8000, etc.), bovine serum albumin, Ficoll, and dextran (e.g., Dextran 6000).

In some embodiments, ligation reaction is carried out at a suitable temperature and reaction time period. In some embodiments, the ligation reaction temperature is from about 2° C. to about 60° C. In some embodiments, the ligation reaction temperature is from 4° C. to 55° C., 4° C. to 50° C., 4° C. to 45° C., or 10° C. to 40° C. In some embodiments, the ligation reaction temperature is 2° C., 5° C., 10° C., 15° C., 20° C., 25° C., 30° C., 37° C., 40° C., 45° C., 50° C., 55° C., or 60° C. In some embodiments, the ligation reaction temperature can use different temperatures, for example a temperature at which stable hybrids are formed between polynucleotide substrate(s) followed by a higher temperature to promote completion of ligation reaction.

In some embodiments, the ligation reaction time can be a sufficient time for ligation of polynucleotide substrate(s). In some embodiments, the ligation reaction time is from 0.5-72 hr or longer. In some embodiments, the ligation reaction time is 1-72 hr, 2-48 hr, or 2-24 hr. In some embodiments, the ligation reaction time is 0.5, 1, 2, 4, 5, 12, 24, 48, or 72 hr or longer.

In some embodiments, the ligase reaction is carried out at a suitable pH. In some embodiments, the pH of the ligase reaction is about pH 5-9, about pH 5.5-8.5, about pH 6-8, or about pH 6.5-7.5. In some embodiments, the pH of the ligase reaction is about pH 5, about pH 5.5, about pH 6, about pH 6.5, about pH 7, about pH 7.5, about pH 8, about pH 8.5 or about pH 9.

In some embodiments, the RNA ligase substrate concentration is at about 0.01-1 mM, 0.05-0.9 mM, 0.1-0.8 mM, 0.2-0.7 mM, or 0.3 mM-0.6 mM. In some embodiments, the RNA ligase substrate is at about 0.01 mM, 0.05 mM, 0.1 mM, 0.2 mM, 0.3 mM, 0.4 mM, 0.5 mM, 0.6 mM, 0.7 mM, 0.8 mM, 0.9 mM or 1 mM. In some embodiments, the RNA ligase substrate concentration is at about 1-10 mM, 2-8 mM, or 4-6 mM. In some embodiments, the engineered RNA ligase exhibits increased product yield at about 1 mM, 2 mM, 3 mM, 4 mM, 5 mM, 6 mM, 7 mM, 8 mM, 9 mM, or 10 mM.

In some embodiments, the polynucleotide substrate is bound to a support, and the ligation reaction carried out with the engineered RNA ligase to ligate a polynucleotide substrate to the support bound polynucleotide, or ligate one or more polynucleotide substrates hybridized to the support-bound polynucleotide substrate.

In some embodiments, the ligation reaction is carried out with an engineered RNA ligase bound to a support. In some embodiments, the polynucleotide substrates in solution can be contacted with the support-bound RNA ligase to ligate the polynucleotide substrates. In some embodiments, the polynucleotide substrates in solution are passed through a bed of support-bound engineered RNA ligase until sufficient ligated product has formed.

In some embodiments, the ligase reaction further includes a nucleotide substrate regeneration system to regenerate co-factor substrate ATP (or dATP). In some embodiments, the ATP recycling or regeneration system includes a nucleoside monophosphate kinase for converting AMP to ADP in presence of a phosphate donor. Various nucleoside monophosphate kinase can be used for the conversion of AMP to ADP, including homologs of nucleoside monophosphate kinases. In some embodiments, more than one nucleoside monophosphate kinase can be used in the regeneration system. In some embodiments, the nucleoside monophosphate kinase is an adenosine monophosphate kinase (e.g., adenylate kinase), cytidine monophosphate (CMP) kinase, uridine monophosphate (UMP) kinase, or guanylate-monophosphate (GMP) kinase.

In some embodiments, a nucleoside monophosphate kinase useful in the enzymatic reactions is a cytidine monophosphate kinases. Various suitable cytidine monophosphate kinases are known in the art. These include homologs of cytidine monophosphate kinases. In some embodiments, a cytidine monophosphate kinase useful in the enzymatic reactions includes, among others, the cytidine monophosphate kinase of Thermus thermophilus (Q5SL35), Pyrococcus furiosus (Q8U2L4), Pseudomonas putida (AF048857.1), Escherichia coli K-12 MG1655 (P0A6I0), Clostridium acetobutylicum (Q97I08), Halobacterium salinarum (Q9HPA5) Bacillus acidicola (WP_066270173), Acetobacter aceti (WP_010667744), Acidithiobacillus thiooxidans (WP_024892761.1), Acidithiobacillus ferrooxidans (WP_064220349.1), Metallosphaera sedula (WP_011921264.1), Amphibacillus xylanus (WP_015009966.1) Thioalkalivibrio denitrificans (WP_077278466.1), Vibrio psychroerythus (Q482G4), Pseudoalteromonas haloplanktis (Q3ILA1), Psychrobacter arcticus (Q4FRL5), Psychromonas ingrahamii (A1 SZ01), Pseudomonas syringae (xQ4ZQ97) and Halobacterium salinarum (Q9HPA5).

In some embodiments, a nucleoside monophosphate kinase useful in the enzymatic reactions is a uridine monophosphate kinase. Various suitable uridine monophosphate kinases are known in the art. These include homologs of uridine monophosphate kinases. In some embodiments, a uridine monophosphate kinase useful in the enzymatic reactions includes, among others, the uridine monophosphate kinase of Pyrococcus furiosus (Q8U122), Thermus thermophilus (P43891), Pseudomonas putida (I7BW46), Escherichia coli K-12 MG1655 (P0A7E9), Aspergillus niger (A2R195), Saccharomyces cerevisiae (P15700), Clostridium acetobutylicum (Q97I64) ATCC 824 PyrH Halobacterium salinarum (Q9HNN8), Picrophilus torridus (WP_048059653), Metallosphaera sedula (WP_012021705), Thermoplasma acidophilum (WP_010900913), Sulfolobus solfataricus (WP_009992427), Acetobacter aceti (WP_042788648), Thioalkalivibrio sp. HK1 (WP_081759172.1), Amphibacillus xylanus (WP_015010200.1), Vibrio psychroerythus (Q485G8), Pseudoalteromonas haloplanktis (Q3IIX6), Psychrobacter arcticus (Q4FRH5), Psychromonas ingrahamii (ABM04676.1), Pseudomonas syringae (Q4ZWS6), and Halobacterium salinarum (Q9HNN8).

In some embodiments, the nucleoside monophosphate kinase useful in the enzymatic reactions is a guanosine monophosphate kinase (guanylate kinase). Various suitable guanylate kinases are known in the art. These include homologs of guanylate kinases. In some embodiments, a guanylate kinase useful in the enzymatic reactions includes, among others, the guanylate kinase of Thermotoga maritima (Q9X215), Thermus thermophilus (Q5SI18), Pseudomonas putida (I7C087), Escherichia coli K-12 (P60546), Aspergillus niger (A2QPV2), Saccharomyces cerevisiae (P15454), Clostridium acetobutylicum (Q97ID0), Acidithiobacillus ferrooxidans (WP_064219869.1), Acidithiobacillus thiooxidans (WP_010637919.1), Bacillus acidicola (WP_066264774.1), Acetobacter aceti (WP_018308252.1), Amphibacillus xylanus (WP_015010280.1), Thioalkalivibrio sulfidiphilus (WP_018953989.1), Vibrio psychroerythus (Q47UB3), Pseudoalteromonas haloplanktis (Q3IJH8), Psychrobacter arcticus (Q4FQY7), Psychromonas ingrahamii (A1T0P1), and Pseudomonas syringae (Q4ZZY8).

In some embodiments, the nucleoside monophosphate kinase useful in the enzymatic reactions is an adenosine monophosphate kinase (adenylate kinase). Various suitable adenylate kinases are known in the art. These include homologs of adenylate kinases. In some embodiments, the adenylate kinase is a bacterial, fungal, plant, or animal adenylate kinase. In some embodiments, an adenylate kinase useful in the enzymatic reactions includes, among others, adenylate kinases of Thermus thermophilus (Q72125), Pyrococcus furiosus (Q8U207), Pseudomonas putida (17CAA9), Escherichia coli K-12 W3110 (P69441), Aspergillus niger CBS 513.88 (A2QPN9), Saccharomyces cerevisiae (P07170), Clostridium acetobutylicum (Q97E39), Halobacterium salinarum (Q9HPAT), Acidithiobacillus thiooxidans (WP_024894015.1), Acidithiobacillus ferrooxidans (WP_064218420.1), Bacillus acidicola (WP_066267988.1), Sulfolobus solfataricus (WP_009991241.1), Saccharomyces cerevisiae (P07170), Thermotoga neapolitana (Q8GGL2), Escherichia coli (P69441) and Geobacillus stearothermophilus (WP_049624206.1). In some embodiments, the adenylate kinase is an engineered adenylate kinase described in International Patent Application No. PCT/US2024/051084, filed Oct. 11, 2024, incorporated herein by reference.

In some embodiments, the ATP regeneration system includes at least an enzyme for the conversion of ADP to ATP in presence of a phosphate donor. In some embodiments, the ATP regeneration system includes, among others, an acetate kinase, pyruvate kinase, creatine kinase, or polyphosphate kinase (see, e.g., Endo et al., Adv. Synth. Catal., 2002, 343:521-526; Andexer et al., Chem Bio Chem., 2015, 16:380-386). In the ATP regeneration system, the phosphate donor for the conversion of ADP to ATP is selected based on the ATP regenerating enzyme employed. By way of example and not limitation, if acetate kinase enzyme is used for conversion of ADP to ATP, the phosphate donor is acetyl-phosphate. If pyruvate kinase is used for the conversion of ADP to ATP, the phosphate donor is phosphoenolpyruvate. If creatine kinase is used for the conversion of ADP to ATP, the phosphate donor is creatine phosphate. If polyphosphate kinase is used for the conversion of ADP to ATP, the phosphate donor is inorganic polyphosphate.

Accordingly, in some embodiments, the ATP regenerating system includes pyruvate kinase and phosphoenolpyruvate. In some embodiments, the ATP regenerating system includes creatine kinase and creatine phosphate. In some embodiments, the ATP regenerating system includes polyphosphate kinase and inorganic polyphosphate. In some embodiments, the ATP regenerating system includes acetate kinase and acetyl phosphate. In some embodiments, the acetate kinase is an acetate kinase of Escherichia coli str. K-12 substr. MG1655 (NP_416799.1), Corynebacterium jeikeium K411 (WP_011272972.1), Lactococcus cremoris subsp. cremoris KW2 (WP_011835968.1), Lactococcus lactis (WP_004254593.1), Marinitoga sp. 38H-ov (WP_165147355.1), Thermotoga sp. KOL6 (WP_101510533.1), Thermosipho melaniensis (WP_012057479.1), Thermotoga sp. RQ7 (WP_041844042.1), and Thermosipho africanus (WP_004102380.1). In some embodiments, the acetate kinase is an engineered acetate kinase described in International Patent Application No. PCT/US2024/051118, filed Oct. 11, 2024, incorporated by reference herein.

### Computer Systems

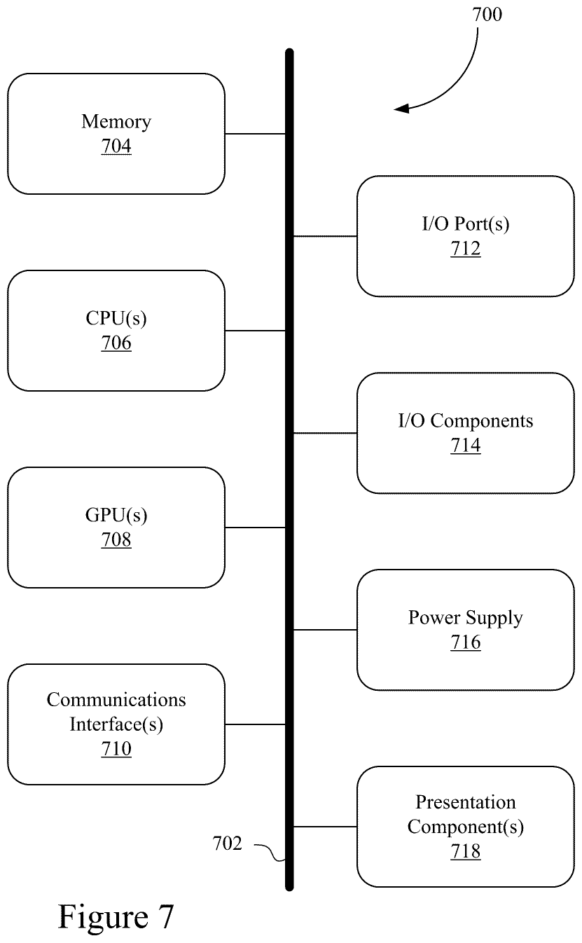

FIG. 7 is a block diagram of an example of the computing device or system 700 suitable for use in implementing computational aspects of some embodiments of the present disclosure. For example, device 700 may be suitable for implementing some or all operations associated with predicting ligation reaction characteristics for ligation sites on a nucleic acid. For example, a computational system such as system 700 may be employed to receive sequence and chemical modification information and/or other input about product nucleic acid, extract features from such input, and predict ligation reaction characteristics for various potential ligation sites, as disclosed herein. In other examples, a computational system such as system 700 may be employed to receive training data about features of product nucleic acids, ligation sites, and ligation reactions at the ligation sites, and use such training data to train a machine learning model that predicts a ligation reaction characteristic based on features.

Computing device 700 may include a bus 702 that directly or indirectly couples the following devices: memory 704, one or more central processing units (CPUs) 706, one or more graphics processing units (GPUs) 708, a communication interface 710, input/output (I/O) ports 712, input/output components 714, a power supply 716, and one or more presentation components 718 (e.g., display(s)). In addition to CPU 706 and GPU 708, computing device 700 may include additional logic devices that are not shown in FIG. 7, such as but not limited to an image signal processor (ISP), a digital signal processor (DSP), a deep learning processor (DLP), an ASIC, an FPGA, or the like.

Although the various blocks of FIG. 7 are shown as connected via the bus 702 with lines, this is not intended to be limiting and is for clarity only. For example, in some embodiments, a presentation component 718, such as a display device, may be considered an I/O component 714 (e.g., if the display is a touch screen). As another example, CPUs 706 and/or GPUs 708 may include memory (e.g., the memory 704 may be representative of a storage device in addition to the memory of the GPUs 708, the CPUs 706, and/or other components). In other words, the computing device of FIG. 7 is merely illustrative. Distinction is not made between such categories as “workstation,” “server,” “laptop,” “desktop,” “tablet,” “client device,” “mobile device,” “hand-held device,” “electronic control unit (ECU),” “virtual reality system,” and/or other device or system types, as all are contemplated within the scope of the computing device of FIG. 7.

Bus 702 may represent one or more busses, such as an address bus, a data bus, a control bus, or a combination thereof. The bus 702 may include one or more bus types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus.

Memory 704 may include any of a variety of computer-readable media. The computer-readable media may be any available media that can be accessed by the computing device 700. The computer-readable media may include both volatile and nonvolatile media, and removable and non-removable media. By way of example, and not limitation, the computer-readable media may comprise computer-storage media and/or communication media.

The computer-storage media may include both volatile and nonvolatile media and/or removable and non-removable media implemented in any method or technology for storage of information such as computer-readable instructions, data structures, program modules, and/or other data types. For example, memory 704 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 700. As used herein, computer storage media does not comprise signals per se.

The communication media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the communication media may include wired media such as a wired network or direct-wired connection, and wireless media such as acoustic, RF, infrared and other wireless media. Combinations of any of the above should also be included within the scope of computer-readable media.

CPU(s) 706 may be configured to execute the computer-readable instructions to control one or more components of the computing device 700 to perform one or more of the methods and/or processes described herein. CPU(s) 706 may each include one or more cores (e.g., one, two, four, eight, twenty-eight, seventy-two, etc.) that are capable of handling a multitude of software threads simultaneously. CPU(s) 706 may include any type of processor and may include different types of processors depending on the type of computing device 700 implemented (e.g., processors with fewer cores for mobile devices and processors with more cores for servers). For example, depending on the type of computing device 700, the processor may be an ARM processor implemented using Reduced Instruction Set Computing (RISC) or an x86 processor implemented using Complex Instruction Set Computing (CISC). Computing device 700 may include one or more CPUs 706 in addition to one or more microprocessors or supplementary co-processors, such as math co-processors.

GPU(s) 708 may be used by computing device 700 to render graphics (e.g., 3D graphics). GPU(s) 708 may include many (e.g., tens, hundreds, or thousands) of cores that are capable of handling many software threads simultaneously. GPU(s) 708 may generate pixel data for output images in response to rendering commands (e.g., rendering commands from CPU(s) 706 received via a host interface). GPU(s) 708 may include graphics memory, such as display memory, for storing pixel data. The display memory may be included as part of memory 704. GPU(s) 708 may include two or more GPUs operating in parallel (e.g., via a link). When combined, each GPU 708 can generate pixel data for different portions of an output image or for different output images (e.g., a first GPU for a first image and a second GPU for a second image). Each GPU can include its own memory or can share memory with other GPUs.

In examples where the computing device 700 does not include the GPU(s) 708, the CPU(s) 706 may be used to render graphics.

Communication interface 710 may include one or more receivers, transmitters, and/or transceivers that enable computing device 700 to communicate with other computing devices via an electronic communication network, included wired and/or wireless communications. Communication interface 710 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet), low-power wide-area networks (e.g., LoRaWAN, SigFox, etc.), and/or the internet.

I/O ports 712 may enable the computing device 700 to be logically coupled to other devices including I/O components 714, presentation component(s) 718, and/or other components, some of which may be built in to (e.g., integrated in) computing device 700. Illustrative I/O components 714 include a microphone, mouse, keyboard, joystick, track pad, satellite dish, scanner, printer, wireless device, etc. I/O components 714 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of computing device 700. Computing device 700 may be include depth cameras, such as stereoscopic camera systems, infrared camera systems, RGB camera systems, touchscreen technology, and combinations of these, for gesture detection and recognition. Additionally, computing device 700 may include accelerometers or gyroscopes (e.g., as part of an inertia measurement unit (IMU)) that enable detection of motion. In some examples, the output of the accelerometers or gyroscopes may be used by computing device 700 to render immersive augmented reality or virtual reality.

Power supply 716 may include a hard-wired power supply, a battery power supply, or a combination thereof. Power supply 716 may provide power to computing device 700 to enable the components of computing device 700 to operate.

Presentation component(s) 718 may include a display (e.g., a monitor, a touch screen, a television screen, a heads-up-display (HUD), other display types, or a combination thereof), speakers, and/or other presentation components. Presentation component(s) 718 may receive data from other components (e.g., GPU(s) 708, CPU(s) 706, etc.), and output the data (e.g., as an image, video, sound, etc.).

The disclosure may be described in the general context of computer code or machine-useable instructions, including computer-executable instructions such as program modules, being executed by a computer or other machine, such as a cell phone, tablet, or other handheld device. Generally, program modules including routines, programs, objects, components, data structures, etc., refer to code configured to perform particular tasks or implement particular data types. The disclosure may be practiced in a variety of system configurations, including hand-held devices, consumer electronics, general-purpose computers, more specialty computing devices, etc. The disclosure may also be practiced in distributed computing environments where tasks are performed by remote-processing devices that are linked through a communications network. Some disclosed embodiments may be implemented, at least in part, using cloud-based resources.

### EXAMPLES

### Example 1

### Data Collections for Single-Nick Ligation Reactions

Three targeting dsRNA molecules were selected to test the single nick designs. The first dsRNA contains 5′-mGmAmCmUmUmUfCmAfUfCfUmGmUmAmAmCmCmAmAmGmA as sense strand and 5′-mUfCmUmUmGfGmUmUfAmCmAmGmAfUmGfAmAmAmGmUmCmCmA as antisense strand; the second dsRNA contains 5′-mCmAmGmAmAmAfGmAfGmUfCmUmGmGmAmAmAmUmAmUmA as sense strand and 5′-mUfAmUmAmUfUmUfCfCmAmGfAmCfUmCfUmUfUmCfUmGmGmU as antisense strand; the third dsRNA contains 5′-mUmGmGmGmAmUfUmUfCfAfUmUfUmUfAmUfCmUmGmAmA as sense strand and 5′-mUmUfCfAfGfAmUfAmAfAfAmUmGfAmAfAmUmCmCmCmAmUmC as antisense strand. With an intact opposing strand, the nick was designed from between 6^{th}/7^{th} nucleotide to between 15^{th}/16^{th} nucleotide (5′ to 3′ direction), and therefore in total (10 antisense strand nick design+10 sense strand nick design)×3 molecules=60 oligo substrate sets were synthesized and tested. In each design, one fragment containing the ligation site is FAM labeled for detection by capillary electrophoresis (CE).

28 variants, including 4 wild type proteins, 4 variants from one lineage, and 22 variants from another lineage, were purified. To initiate the ligation reaction, 0.1 mg/mL of purified ligase was added to a mixture containing 1.25 mM of each of the three oligo substrate, 70 mM of Tris-HCl buffer at pH 8.0, 30 mM MgCl₂, 6 mM ATP. Nanoliter scale liquid transfers were carried out by Echo Acoustic Liquid Handler. 150 nanoliter of the reactions were carried out in a 384-well Bio-rad PCR plate, which was mixed by brief vortex, incubated for 16 hours at 33° C. in a PCR thermocycler, quenched by adding 17.5 mM EDTA, and diluted for analytical analysis by CE. Using the peak area from CE analytical method, the % conversion for each sample was calculated as shown below:

$$\% conversion = \frac{area under the curve for the product peak}{\begin{matrix}Total area under the curve \\ for all products and remaining fragments\end{matrix}} * 100$$

### Example 2

### Model Construction for Single-Nick Ligation Reactions

Single strand RNA region of the dsRNA was removed for featurization, and only the 21-nucleotide long dsRNA region was considered. 2-methyl or 2-fluoro modifications were encoded.

Various region of dsRNA sequence centered from the nick position, including [−2, +2], [−6, +5], [−3, +3], [−3, +2], and [−6, +2], along with the corresponding opposing strand sequences and modifications, were tested to obtain optimal featurization for single nick designs. Full length 21 dsRNA region along with the corresponding opposing strand sequences and modifications was also tested.

The position of the nick was represented as the distance from the 5′ end of a given strand, the distance from the 3′ end of the strand, and as the size of the shortest RNA fragment (the minimum of the 2 prior features).

Six model types, Ridge, Lasso, GPR (Gaussian Process Regression), PLS (Partial Least Squares), Random Forest, SVM (Support Vector Machine), were evaluated in regression and binary classification modes. Models were trained for each enzyme individually. Binary classification models utilized an activity threshold of 85%. Models were built in python utilizing publicly available packages such as sklearn.

Data from the first two dsRNA molecules listed in Example 1 were used for model training and cross validations and ultimately evaluated on the third dsRNA molecule. Data was scaled prior to model training and the model was trained on averages of the triplicate screening data.

### Example 3

### Data Collections for Double-Nick Ligation Reactions

Four targeting dsRNA molecules were selected to test the double nick designs. The first, second, and third targeting dsRNA molecules are the same as the three in Example 1, and the fourth one contains 5′-mAmUfGmUfUmGmUfCfCfUmUmUmUmGmCmUmUmUmUmGmU as sense strand and 5′-mAfCmAfAfAfAmGfCmAfAmAmAmGmGfAmCfAmAfCmAmUmGmU as antisense strand. One nick was designed from between 6^{th}/7^{th} nucleotide to between 15^{th}/16^{th} nucleotide (5′ to 3′ direction) on one strand, and the other nick was designed from between 6^{th}/7^{th} nucleotide to between 15^{th}/16^{th} nucleotide (5′ to 3′ direction) on the opposing strand, and blunt end dsRNA fragment nick designs were avoided. In each design, one fragment containing the ligation site is FAM labeled for detection by CE. Therefore, in total 10 sense strand nick design x 9 antisense strand nick design x 4 molecules=360 oligo substrate sets were synthesized and tested.

Two top variants from Example 1, Rd17BB and Arwd8.001, were used here in double-nick ligation reactions. To initiate the ligation reaction, 0.1 mg/mL of purified ligase was added to a mixture containing 1.25 mM of each of the two FAM labeled oligos, 1.66 mM of each of the two non-labeled oligos, 70 mM of Tris-HCl buffer at pH 8.0, 30 mM MgCl₂, 6 mM ATP. Nanoliter scale liquid transfers were carried out by Echo Acoustic Liquid Handler. 150 nanoliter of the reactions were carried out in a 384-well Bio-rad PCR plate, which was mixed by brief vortex, incubated for 16 hours at 33° C. in a PCR thermocycler, quenched by adding 17.5 mM EDTA, and diluted for analytical analysis by CE. Using the peak area from CE analytical method, the % conversion for each strand of each sample was calculated as shown below:

$$\% conversion = \frac{area under the curve for the product peak}{\begin{matrix}Total area under the curve \\ for all products and remaining fragments\end{matrix}} * 100$$

### Example 4

### Model Construction for Double-Nick Ligation Reactions

Single strand RNA region of the dsRNA was removed for featurization, and only the 21-nucleotide long dsRNA region was considered. 2-methyl or 2-fluoro modifications were encoded with binary encoding. Bases A (adenine), C (cytosine), G (guanine), U (uradine) were encoded.

Various region of dsRNA sequence centered from the nick position, including [−2, +2], [−6, +5], and [−3, +3], along with the corresponding opposing strand sequences and modifications, were tested to obtain optimal featurization for double nick designs.

The position of the nicks were represented as the distance from the 5′ end of a given strand, the distance from the 3′ end of the strand, and as the size of the shortest RNA fragment (the minimum of the 2 prior features). The positions of two nicks relative to each other were represented as two features: the distance between them in terms of number of bases and the “sign”, which represents whether the other nick is on the 3′ side (1) or the 5′ side (0) of its own strand.

Six model types, Ridge, Lasso, GPR (Gaussian Process Regression), PLS (Partial Least Squares), Random Forest, SVM (Support Vector Machine), were evaluated in regression and binary classification modes. Models were trained for each enzyme individually. Binary classification models utilized an activity threshold of 85%. Models were built in python utilizing publicly available packages such as sklearn.

Data from the first three dsRNA molecules listed in Examples 1 and 3 were used for model training and cross validations and ultimately evaluated on the forth dsRNA molecule in Example 3. We tested six featurization modes, including “Mode A” using 2D nucleotide encoding and [−2, +2] region, “Mode B” using 4D nucleotide encoding and [−2, +2] region, “Mode C” using 2D nucleotide encoding and [−6, +5] region, “Mode D” using 4D nucleotide encoding and [−6, +5] region, “Mode E” using 2D nucleotide encoding and [−3, +3] region, “Mode F” using 4D nucleotide encoding and [−3, +3] region. Data was scaled prior to model training and the model was trained on averages of the triplicate screening data.

### CONCLUSION

Although the foregoing embodiments have been described in some detail for purposes of clarity of understanding, it will be apparent that certain changes and modifications may be practiced within the scope of the appended claims. It should be noted that there are many alternative ways of implementing the processes, systems, and apparatus of the present embodiments. Accordingly, the present embodiments are to be considered as illustrative and not restrictive, and the embodiments are not to be limited to the details given herein.

What is claimed is:

1. A computer-implemented method comprising:
receiving structural information charactering a double stranded nucleic acid, wherein the structural information comprises a nucleotide sequence of the double stranded nucleic acid and one or more chemical modifications of the nucleotides of the double stranded nucleic acid;
identifying multiple ligation sites on one or both strands of the double stranded nucleic acid;
for each of the multiple ligation sites,
(a) identifying features characterizing a current ligation site of the double stranded nucleic acid, wherein the features comprise information about the types of nucleotides located at first defined positions with respect to the current ligation site and/or information about one or more chemical modifications of nucleotides located at second defined positions with respect to the current ligation site, and
(b) using a trained computational model to predict, based on the features of the current ligation site, whether the current ligation site meets a criterion for a ligation reaction at the current ligation site with a ligase; and
based on operation (b), identifying a combination of ligation sites to produce the double stranded nucleic acid using the ligase.

2. The method of claim 1, wherein the double stranded nucleic acid is an RNA molecule.

3. The method of claim 2, wherein the RNA molecule has length of about 10 to 50 nucleotides.

4. The method of claim 2, wherein the RNA molecule is an siRNA molecule.

5. The method of claim 4, wherein the siRNA molecule is a therapeutic siRNA molecule.

6. The method of claim 1, wherein the double stranded nucleic acid comprises a hairpin structure.

7. The method of claim 1, wherein the double stranded nucleic acid comprises a single stranded portion.

8. The method of claim 1, wherein the combination of ligation sites defines a combination of nucleic acid fragments for producing the double stranded nucleic acid, and wherein the longest nucleic acid fragment in the combinations of nucleic acid fragments has length of no greater than about 30 nucleotides.

9. The method of claim 8, wherein the combination of nucleic acid fragments has a total of 2 to 10 nucleic acid fragments.

10. The method of claim 1, wherein the features characterizing the current ligation site of the double stranded nucleic acid further comprise a separation distance between the current ligation site on a first strand of the double stranded nucleic acid and a second ligation site on a second strand of the double stranded nucleic acid.

11. The method of claim 1, wherein the information about the types of nucleotides located at the first defined positions comprises (a) whether the nucleotide comprises a purine or pyrimidine, and (b) the base pair matches.

12. The method of claim 1, wherein the first defined positions with respect to the current ligation site are within ten nucleotides upstream and/or downstream of the ligation site.

13. The method of claim 1, wherein the information about the one or more chemical modifications of nucleotides located at the second defined positions comprises an indication of whether a chemical modification to a sugar moiety is present.

14. The method of claim 13, wherein the chemical modification to the sugar moiety comprises a substitution on the 2-position of a ribose.

15. The method of claim 14, wherein the substitution on the 2 position of a ribose comprises a 2-F substitution or a 2-OMe substitution.

16. The method of claim 1, wherein the information about the one or more chemical modifications of nucleotides located at the second defined positions comprises replacing a phosphate group with a phosphate derivative.

17. The method of claim 1, wherein the second defined positions with respect to the current ligation site are within ten nucleotides upstream and/or downstream of the ligation site.

18. The method of claim 1, wherein the criterion for the ligation reaction is a predicted efficiency of the ligation reaction at the current ligation site.

19. The method of claim 1, wherein the criterion for the ligation reaction is a predicted a probability that the ligation reaction will have at least defined minimum efficiency.

20. The method of claim 1, further comprising ranking multiple combinations of ligation sites and ligases that meet the criterion for the ligation reaction at the combinations of ligation sites.

21. The method of claim 1, wherein the method does not identify any combination of ligation sites that will require a blunt end ligation.

22. The method of claim 1, wherein the method is constrained to select only combinations of ligation sites in which a single ligase can meet the criterion at all ligation sites.

23. The method of claim 1, wherein the trained computational model comprises a regression model.

24. The method of claim 1, wherein the trained computational model comprises a machine learning classification model.

25. The method of claim 1, wherein the trained computational model comprises an ensemble of machine learning models.

26. The method of claim 1, wherein the criterion for the ligation reaction comprises a minimum probability that the ligation reaction will have at least defined minimum efficiency at each of the ligation sites in the combination of ligation sites.

27. A method of synthesizing a double stranded nucleic acid, the method comprising:
for each of the multiple ligation sites on one or both strands of the double stranded nucleic acid, computationally generating features characterizing a current ligation site, wherein the features comprise information about the types of nucleotides located at first defined positions with respect to the current ligation site and/or information about one or more chemical modifications of nucleotides located at second defined positions with respect to the current ligation site; and
using a trained computational model to identify, based on the features for each of the multiple ligation sites, a combination of ligation sites and, optionally, a ligase for ligating a plurality of nucleic acid fragments at the combination of ligation sites;
chemically synthesizing the fragments; and
ligating the chemically synthesized fragments with the ligase to produce the double stranded nucleic acid.

28. The method of claim 27, wherein chemically synthesizing the fragments comprises a solid phase synthesis.

29. The method of claim 28, wherein the solid phase synthesis employs a phosphoramidite chemistry.

30. The method of claim 27, wherein the ligating is performed in the presence of a nucleotide cofactor or substrate used to catalyze a ligation reaction.

31. The method of claim 27, wherein the ligating is performed with an engineered RNA ligase.

32. The method of claim 27, wherein the double stranded nucleic acid is an RNA molecule.

33. The method of claim 32, wherein the RNA molecule has length of about 10 to 50 nucleotides.

34. The method of claim 32, wherein the RNA molecule is an siRNA molecule.

35. The method of claim 27, wherein, among the plurality of nucleic acid fragments, the longest nucleic acid fragment has length of no greater than about 30 nucleotides.

36. The method of claim 27, wherein the plurality of nucleic acid fragments has a total of 2 to 10 nucleic acid fragments.

37. The method of claim 27, wherein the information about the types of nucleotides located at the first defined positions comprises (a) whether the nucleotide comprises a purine or pyrimidine, and (b) the base pair matches.

38. The method of claim 27, wherein the first defined positions with respect to the current ligation site are within ten nucleotides upstream and/or downstream of the ligation site.

39. The method of claim 27, wherein the information about the one or more chemical modifications of nucleotides located at the second defined positions comprises an indication of whether a chemical modification to a sugar moiety is present.

40. The method of claim 39, wherein the chemical modification to the sugar moiety comprises a substitution on the 2-position of a ribose.

41. The method of claim 40, wherein the substitution on the 2 position of a ribose comprises a 2-F substitution or a 2-OMe substitution.

42. The method of claim 27, wherein the second defined positions with respect to the current ligation site are within ten nucleotides upstream and/or downstream of the ligation site.

43. The method of claim 27, further comprising using the trained computational model to output a probability that the ligation reaction will have at least defined minimum efficiency.

44. The method of claim 27, wherein the trained computational model is a machine learning model.

45. A method of training a machine learning model for predicting one or more characteristics of ligation reaction, the method comprising:
receiving training data for each of a plurality of ligation reactions, wherein the training data for each ligation reaction comprises (a) ligation site for a product nucleic acid molecule, (b) a plurality features characterizing nucleotide types and/or chemical modifications to nucleotides proximate the ligation site, and (c) a characteristic of the ligation reaction at the ligation site; and
training the machine learning model using the training data, wherein the machine learning model is configured to predict the characteristic of the ligation reaction based on, at least, the plurality features characterizing nucleotide types and/or chemical modifications to nucleotides proximate the ligation site.

46. The method of claim 45, wherein the training comprises a regression technique.

47. The method of claim 45, wherein the training comprises a regularization technique.

48. The method of claim 45, wherein the product nucleic acid molecule is an RNA molecule.

49. The method of claim 48, wherein the RNA molecule has length of about 10 to 50 nucleotides.

50. The method of claim 48, wherein the RNA molecule is an siRNA molecule.

51. The method of claim 45, wherein the plurality features comprises a separation distance between a first ligation site on a first strand of a double stranded nucleic acid and a second ligation site on a second strand of the double stranded nucleic acid.

52. The method of claim 45, wherein the plurality features characterizing nucleotide types comprises (a) whether the nucleotide comprises a purine or pyrimidine, and (b) the base pair matches.

53. The method of claim 45, wherein the plurality features characterizing chemical modifications to nucleotides comprises an indication of whether a chemical modification to a sugar moiety is present.

54. The method of claim 53, wherein the chemical modification to the sugar moiety comprises a substitution on the 2-position of a ribose.

55. The method of claim 54, wherein the substitution on the 2 position of a ribose comprises a 2-F substitution or a 2-OMe substitution.

56. The method of claim 45, wherein the plurality features characterizing chemical modifications to nucleotides comprises replacing a phosphate group with a phosphate derivative.

57. The method of claim 45, wherein the characteristic the ligation reaction at the ligation site is an efficiency of the ligation reaction at the ligation site.
